(19) United States
(12) Patent Application Publication
(10) Pub. No.: US 2007/0078506 A1
(43) Pub. Date: Apr. 5, 2007

(54) Method and apparatus for decompressing aneurysms

(76) Inventors: Paul McCormick, Laguna Niguel, CA (US); Stefan Schreck, Fallbrook, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/580,201

(22) Filed: Oct. 12, 2006

Related U.S. Application Data
(63) Continuation-in-part of application No. 11/104,303, filed on Apr. 12, 2005.
(60) Provisional application No. 60/590,870, filed on Jul. 23, 2004.
(60) Provisional application No. 60/561,852, filed on Apr. 13, 2004.
(60) Provisional application No. 60/726,299, filed on Oct. 12, 2005.

(51) Int. Cl. A61F 2/06

(52) U.S. Cl. 623/1.11

(57) ABSTRACT

A method of treating an aneurysm comprising placing an aspiration catheter in communication with the aneurysm. A prosthesis is deployed across an opening to the aneurysm to isolate at least a portion of the aneurysm. Material is aspirated from the aneurysm. Embolizing or other material may be introduced into the aneurysm.

### PRIORITY INFORMATION

This application is a continuation-in-part of U.S. patent Ser. No. 11/104,303, filed Apr. 12, 2005, which claims the benefit of U.S. Provisional Application No. 60/590,870, filed Jul. 23, 2004 and U.S. Provisional Application No. 60/561,852, filed Apr. 13, 2004, this application also claims the benefit of U.S. Provisional Application No. 60/726,299 filed on Oct. 12, 2005, the entire contents of these applications are hereby incorporated by reference herein.

### BACKGROUND OF THE INVENTION

1. Field of the Invention

The present invention relates to method and devices for treating an aneurysm, and in particular, to method and devices for treating abdominal aortic aneurysms.

2. Description of the Related Art

An abdominal aortic aneurysm is a sac caused by an abnormal dilation of the wall of the aorta, a major artery of the body, as it passes through the abdomen. The abdomen is that portion of the body which lies between the thorax and the pelvis. It contains a cavity, known as the abdominal cavity, separated by the diaphragm from the thoracic cavity and lined with a serous membrane, the peritoneum. The aorta is the main trunk, or artery, from which the systemic arterial system proceeds. It arises from the left ventricle of the heart, passes upward, bends over and passes down through the thorax and through the abdomen to about the level of the fourth lumbar vertebra, where it divides into the two common iliac arteries.

The aneurysm usually arises in the infrarenal portion of the diseased aorta, for example, below the kidneys. When left untreated, the aneurysm may eventually cause rupture of the sac with ensuing fatal hemorrhaging in a very short time. High mortality associated with the rupture led initially to transabdominal surgical repair of abdominal aortic aneurysms. Surgery involving the abdominal wall, however, is a major undertaking with associated high risks. There is considerable mortality and morbidity associated with this magnitude of surgical intervention, which in essence involves replacing the diseased and aneurysmal segment of blood vessel with a prosthetic device which typically is a synthetic tube, or graft, usually fabricated of Polyester, Urethane, DACRON®, TEFLON®, or other suitable material.

To perform the surgical procedure requires exposure of the aorta through an abdominal incision which can extend from the rib cage to the pubis. The aorta must be closed both above and below the aneurysm, so that the aneurysm can then be opened and the thrombus, or blood clot, and arteriosclerotic debris removed. Small arterial branches from the back wall of the aorta are tied off. The DACRON® tube, or graft, of approximately the same size of the normal aorta is sutured in place, thereby replacing the aneurysm. Blood flow is then reestablished through the graft. It is necessary to move the intestines in order to get to the back wall of the abdomen prior to clamping off the aorta.

If the surgery is performed prior to rupturing of the abdominal aortic aneurysm, the survival rate of treated patients is markedly higher than if the surgery is performed after the aneurysm ruptures, although the mortality rate is still quite high. If the surgery is performed prior to the aneurysm rupturing, the mortality rate is typically slightly less than 10%. Conventional surgery performed after the rupture of the aneurysm is significantly higher, one study reporting a mortality rate of 66.5%. Although abdominal aortic aneurysms can be detected from routine examinations, the patient does not experience any pain from the condition. Thus, if the patient is not receiving routine examinations, it is possible that the aneurysm will progress to the rupture stage, wherein the mortality rates are significantly higher.

Disadvantages associated with the conventional, prior art surgery, in addition to the high mortality rate include the extended recovery period associated with such surgery; difficulties in suturing the graft, or tube, to the aorta; the loss of the existing aorta wall and thrombosis to support and reinforce the graft; the unsuitability of the surgery for many patients having abdominal aortic aneurysms; and the problems associated with performing the surgery on an emergency basis after the aneurysm has ruptured. A patient can expect to spend from one to two weeks in the hospital after the surgery, a major portion of which is spent in the intensive care unit, and a convalescence period at home from two to three months, particularly if the patient has other illnesses such as heart, lung, liver, and/or kidney disease, in which case the hospital stay is also lengthened. The graft must be secured, or sutured, to the remaining portion of the aorta, which may be difficult to perform because of the thrombosis present on the remaining portion of the aorta. Moreover, the remaining portion of the aorta wall is frequently friable, or easily crumbled.

Since many patients having abdominal aortic aneurysms have other chronic illnesses, such as heart, lung, liver, and/or kidney disease, coupled with the fact that many of these patients are older, the average age being approximately 67 years old, these patients are not ideal candidates for such major surgery.

More recently, a significantly less invasive clinical approach to aneurysm repair, known as endovascular grafting, has been developed. Parodi, et al. provide one of the first clinical descriptions of this therapy. Parodi, J. C., et al., “Transfemoral Intraluminal Graft Implantation for Abdominal Aortic Aneurysms,” 5 Annals of Vascular Surgery 491 (1991). Endovascular grafting involves the transluminal placement of a prosthetic arterial graft within the lumen of the artery.

In general, transluminally implantable prostheses adapted for use in the abdominal aorta comprise a tubular wire cage surrounded by a tubular PTFE or Dacron sleeve. Both balloon expandable and self expandable support structures have been proposed. Endovascular grafts adapted to treat both straight segment and bifurcation aneurysms have also been proposed.

When an abdominal aorta aneurysm is treated with an endovascular graft, the aneurysm should stabilize or shrink. However, in some cases, there is persistent flow of blood into the aneurysm following placement of the graft. Such flow is often referred to as an “endoleak”. Endoleaks can cause continued pressurization of the aneurysm sac, which may leave the patient at risk for abdominal aortic aneurysm rupture, if not resolved or left untreated. Endoleaks are typically due to incomplete sealing, or exclusion of the aneurysm sac or vessel, and/or reflux of blood flow into the sac.

Thus, notwithstanding the many advances which have been made in recent years in the treatment of abdominal aortic aneurysms with grafts, there remains a need for improved methods and devices for reducing and/or preventing endoleaks which may lead to abdominal aortic aneurysm rupture.

### SUMMARY OF THE INVENTION

The present invention relates to a method of treating an aneurysm. In the method, an aspiration catheter is placed in communication with the aneurysm. A prosthesis is deployed across an opening to the aneurysm to isolate at least a portion of the aneurysm. Material is aspirated from the aneurysm. In certain modified embodiments, an agent is introduced into the isolated portion of the aneurysm. In such embodiments, the agent may comprise an embolization material.

In accordance with another aspect of the present invention, there is provided a method of treating a patient. The method comprises the steps of identifying a vascular aneurysm, and positioning a prosthesis across the aneurysm to isolate at least a portion of the aneurysm from an adjacent vessel. Material is thereafter removed from the isolated portion of the aneurysm.

The removing step may be accomplished through a transluminally placed catheter. Alternatively, the removing step may be accomplished through a percutaneous tissue tract.

The removing step may comprise removing at least about 5 cc of blood, and, in certain applications, at least about 10 cc of blood. The method may additionally comprise the step of introducing a media into the isolated portion of the aneurysm, such as an embolization material and/or a drug.

Further features and advantages of the present invention will become apparent to those of skill in the art in view of the detailed description of preferred embodiments which follows, when considered together with the attached drawings and claims.

### BRIEF DESCRIPTION OF THE DRAWINGS

FIG. 1 is a side elevational schematic cross-section of an over the wire aspiration catheter in accordance with one embodiment of the present invention.

FIG. 1A is a cross section taken along the line 1A-1A in FIG. 1.

FIG 1B is a cross-sectional view as in FIG. 1A of a modified embodiment of an aspiration catheter.

FIG. 2A is a side elevational view of an embodiment of a dual lumen aspiration catheter in accordance another embodiment the present invention.

FIG. 2B is a cross section taken along the line 2B-2B in FIG. 2A.

FIG. 2C is a cross-sectional view as in FIG. 2B of a modified embodiment of an aspiration catheter.

FIG. 3 is a schematic representation of the abdominal aorta anatomy with an abdominal aortic aneurysm.

FIG. 4 is a schematic representation as in FIG. 3 of the abdominal aorta anatomy with a bifurcated graft deployed to isolate at least a portion of the aneurysm and an aspiration catheter positioned to aspirate material from the aneurysm.

FIG. 5 is a schematic representation as in FIG. 4 of the abdominal aorta anatomy with the bifurcated graft deployed and after aspirating material from the aneurysm.

FIG. 6 is a schematic representation as in FIG. 5 of the abdominal aorta anatomy with the aspiration catheter removed.

FIG. 7 is a schematic representation as in FIG. 3 of the abdominal aorta anatomy with a straight graft deployed to isolate at least a portion of the aneurysm and an aspiration catheter positioned to aspirate material from the aneurysm.

FIG. 8 is a schematic representation as in FIG. 7 of the abdominal aorta anatomy and after aspirating material from the aneurysm and removing the aspiration catheter.

FIG. 9 is a schematic representation of an exemplary bifurcated vascular prosthesis useful with an embodiment of the present invention showing a main body and branch sections.

FIG. 9A is a schematic representation of an exemplary wire support structure for the bifurcated vascular prosthesis of FIG. 9 showing a main body support structure and detached branch support structures.

FIG. 10 is a schematic representation of the wire support structure as shown in FIG. 9A, illustrating sliding articulation between the branch supports and the main body support.

FIG. 11 is a plan view of a formed wire useful for rolling about an axis to form a branch support structure in accordance with the embodiment shown in FIG. 9A.

FIGS. 12A, 12B and 12C are enlargements of the apexes delineated by lines A, B and C respectively in FIG. 11.

FIG. 13 is a side elevational cross-section of a bifurcation graft delivery catheter useful for introducing a bifurcation graft along the guidewires placed by the dual lumen access catheter of the present invention.

FIG. 14 is an enlargement of the portion delineated by the section 14 in FIG. 13.

FIG. 15 is a cross-section taken along the line 15-15 in FIG. 14.

FIG. 16 is a cross-section taken along the line 16-16 in FIG. 14.

FIG. 17 is a schematic representation of a bifurcated graft deployment catheter positioned within the ipsilateral iliac and the aorta, with an aspiration catheter extending through the contralateral iliac.

FIG. 18 is a schematic representation as in FIG. 17, with the outer sheath proximally retracted and the compressed iliac branches of the graft moving into position within the iliac arteries.

FIG. 19 is a schematic representation as in FIG. 18, with the compressed iliac branches of the graft within the iliac arteries, and the main aortic trunk of the graft deployed within the aorta.

FIG. 20 is a schematic representation as in FIG. 19, with the contralateral iliac branch of the graft deployed.

FIG. 21 is a schematic representation as in FIG. 20, following deployment of the ipsilateral branch of the graft.

FIG. 22 is a schematic representation as in FIG. 21, following aspiration of the aneurysm and reduction of the aneurysm sac.

FIG. 23 is a schematic representation as in FIG. 22 following aspiration of the aneurysm and the injection of an embolization agent removal of the aspiration catheter.

FIG. 24 is a schematic representation as in FIG. 22 following aspiration of the aneurysm and removal of the aspiration catheter.

FIG. 25 is a side view of a syringe member that may be used in combination with an aspiration catheter.

FIG. 26 is a side elevational view of an embodiment of an introducer catheter in accordance an embodiment the present invention.

FIG. 27A is a schematic representation of a bifurcated graft and catheter positioned an aneurysm.

FIG. 27B is a schematic representation of a bifurcated graft and catheter as in FIG. 27A with the catheter proximally withdrawn to a second position.

FIG. 27C is a schematic representation of a bifurcated graft and catheter as in FIG. 27A with the catheter proximally withdrawn to a third position.

### DETAILED DESCRIPTION OF THE PREFERRED EMBODIMENTS

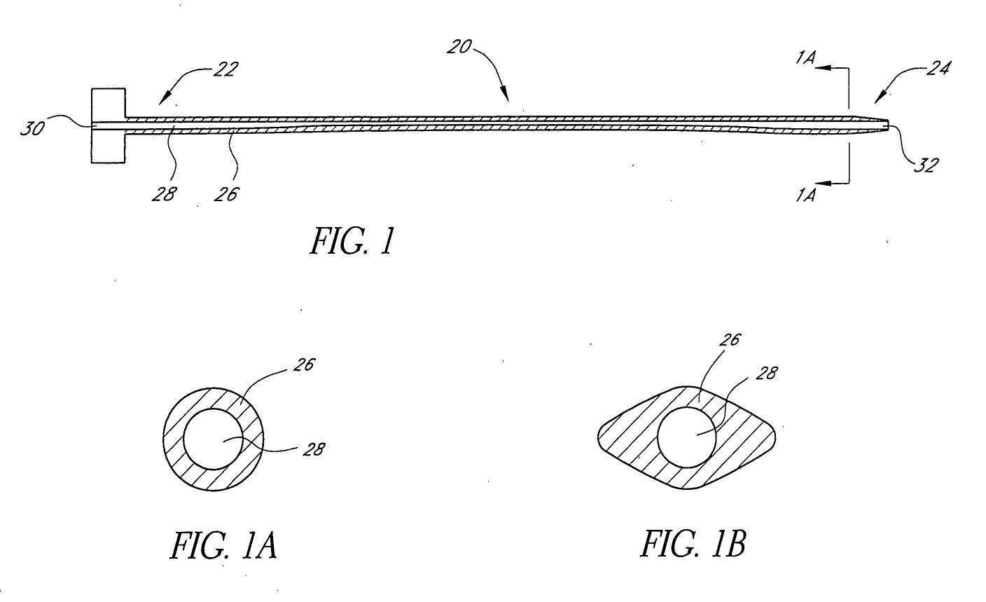

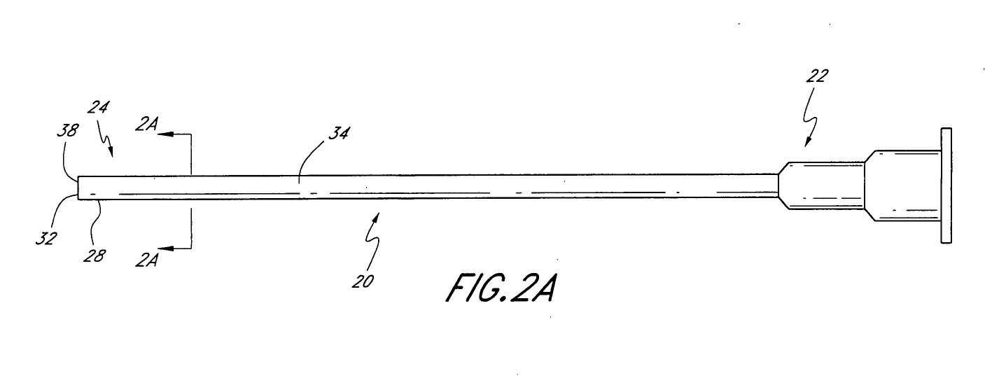

Referring to FIG. 1, there is illustrated an exemplary embodiment of an aspiration catheter 20. The catheter 20 comprises a proximal end 22, a distal end 24 and an elongate flexible tubular body 26 extending therebetween. An aspiration lumen 28 extends axially through the tubular body 26 between a proximal access port 30 and a distal access port 32.

As will be explained in detail below, in one embodiment of use, the aspiration catheter 20 may be used to aspirate blood and possibly thrombus and debris from an aneurysm that has been isolated from the parent vessel by a graft. The aneurysm may be located generally at or near the bifurcation of the lower abdominal aorta and the ipsilateral and contralateral iliac arteries. In such an embodiment, the aneurysm may be isolated by an expandable straight or bifurcated graft. However, the devices and methods disclosed herein are readily adaptable to treat other aneurysms located elsewhere in the body as will be apparent to those of skill in the art in view of the disclosure herein.

The tubular body 26 may be formed in any of a variety of manners which are well known in the art of catheter body manufacturing, such as by extrusion. Suitable extrudable materials include high density polyethylene, medium density polyethylene and other polyethylene blends, nylon, PEBAX, PEEK and others well known in the art. Reinforced tubular bodies may be produced by including a braided layer in or on the wall. The braided wall may comprise any of a variety of materials such as stainless steel, Nitinol, composite fibers and others known in the art. Additional details concerning the tubular body 26 will be recited below.

The aspiration lumen 28 may have an inside diameter of at least about 0.038″ to accommodate a standard 0.035″ diameter guidewire, which can be used to position the catheter 20. Other inside diameters for first lumen 28 can readily be provided, based upon the desired guidewire diameter and desired aspiration flow rate. The tubular body 26 may have a variety of lengths and outside diameters depending upon the application. For aspirating an aneurysm located generally at or near the bifurcation of the lower abdominal, the tubular body 26 generally has a length of within the range of from about 100 cm to about 140 cm and an outside diameter within the range of from about 0.020″ to about 0.25″.

The proximal access port of the catheter 20 may be connected to an aspiration device 31 such as a syringe, pump or other vacuum source such that blood or other material near the distal access port 32 may be withdrawn into the catheter 20 to depressurize the isolated aneurysm.

FIG. 1A illustrates a cross-section through the distal end 24 of the catheter 20 taken at line 1A-1A. As shown in FIG. 1A, the distal end 24 may have a generally circular cross-section. FIG. 1B illustrates a modified embodiment in which the distal end has a generally tapered (e.g., elliptical or oval) cross-sectional shape. As will be explained in more detail below, a generally tapered shape may advantageously reduce leakage between the graft and the aspiration catheter 20 when the catheter 20 is placed on the outside of the graft with the distal end 24 placed in the aneurysm. The outside diameter of the catheter 20 may be tapered along its length, or only in the vicinity of the distal end 24.

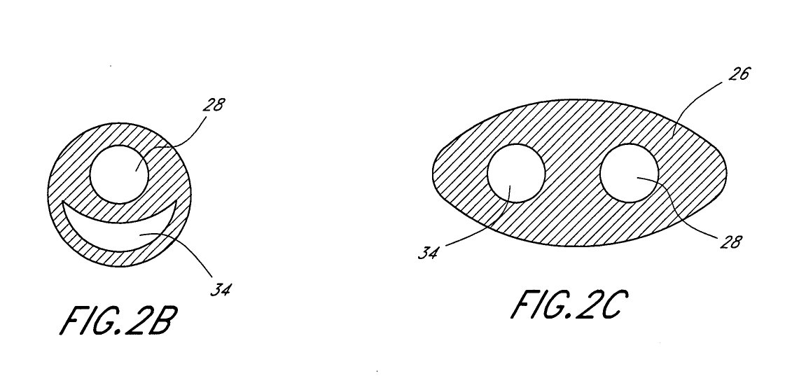

FIGS. 2A and 2B illustrate a modified embodiment of an aspiration catheter 20′. As with the previous embodiment, the catheter 20 comprises a proximal end 22, a distal end 24 and an elongate flexible tubular body 26 extending therebetween. The tubular body 26 is provided with an aspiration lumen 28, extending axially therethrough between a proximal access port (not shown) and a distal access port 32. This embodiment is also provided with a second lumen 34 that extends throughout at least a portion of the tubular body 26, between a proximal port (not shown) and a distal port 38. In this manner, the catheter 20′ is a dual lumen catheter in which the second lumen 34 may be used by a guidewire and the first lumen 26 may be used for aspiration and/or infusion of thrombolytics or other medications, agents or media.

As shown in FIGS. 2B and 2C, the distal end 24 of the catheter 20′ may have a generally circular cross-section (FIG. 2B) or a generally tapered (e.g., elliptical or oval) cross-section (FIG. 2C). As with the previous embodiment, the catheter 20′ may be tapered only at its distal end 24.

The distal end 24 of the catheter 20, 20′ may also be provided with more than one opening or ports to minimize or reduce clotting of the distal aspiration port by loose thrombus, debris etc. during suction. In other embodiments, the distal end of the catheter may be porous. The distal end 24 of the catheter 20 preferably also includes a radiopaque marker 33 as shown in FIGS. 1 and 2A.

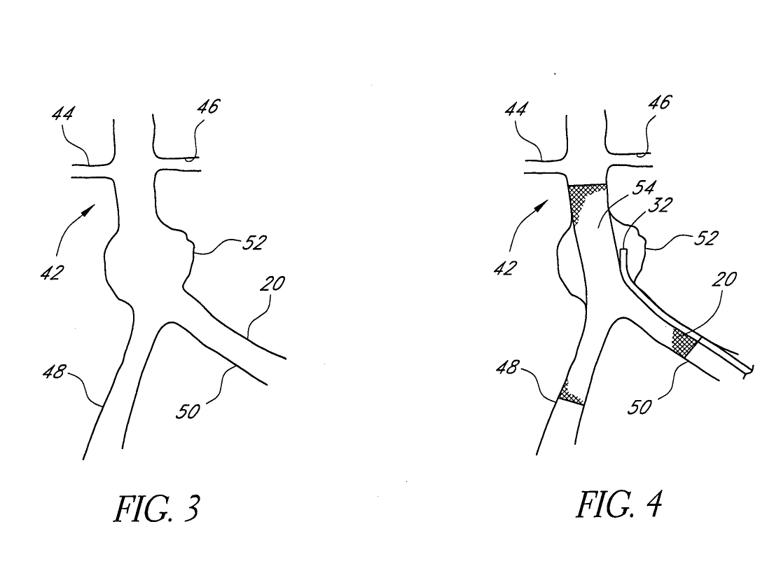

Embodiments of using the aspiration catheter 20 will be described in connection with FIGS. 3 through 8. With initial reference to FIG. 3, there is disclosed a schematic representation of the abdominal part of the aorta and its principal branches. In particular, the abdominal aorta 42 is characterized by a right renal artery 44 and left renal artery 46. The large terminal branches of the aorta are the right and left common iliac arteries 48 and 50. Additional vessels (e.g. second lumbar, testicular, inferior mesenteric, middle sacral) have been omitted for simplification. An abdominal aortic aneurysm 52 is illustrated in the infrarenal portion of the diseased aorta.

With reference to FIG. 4, a bifurcated graft 54, an example of which will be described in more detail below, has been deployed to isolate the aneurysm 52. In addition, the distal access port 32 of the aspiration lumen has been position such that it is in communication with the aneurysm 52 outside of the graft 564. As will be explained in more detail below, the aspiration catheter 20 is preferably positioned in the aneurysm 52 before the bifurcated graft 54 is deployed such that as the graft 54 is deployed, the catheter 20 extends along the outside of the deployed graft 54 and into the aneurysm 52. In positioning the distal end 24, the radiopaque marker 33 can be used in combination with a visualization technique (e.g., an aortogram and/or sacogram) to properly position the distal end 24 within the aneurysm 52.

The aspiration catheter 20 may be advanced transluminally through the contralateral iliac 50 and into the aneurysm 52, as illustrated. Alternatively, the aspiration catheter 20 may be advanced transluminally through the ipislateral iliac 48, and into the aneurysm. The aspiration catheter may further be introduced into the vasculature at a point superior to the aneurysm, and advanced transluminally in an inferior direction through the thoracic and abdominal aorta into the aneurysm. In general, Applicants presently prefer introduction of the aspiration catheter 20 through the contralateral iliac as illustrated.

As a further alternative, aspiration of the isolated aneurysm can be accomplished through a separate percutaneous tissue tract, formed through the chest wall. Once it appears that the deployed graft has sufficiently isolated an aneurysm, a hollow aspiration needle can be introduced directly into the aneurysm sac and utilized to aspirate fluid much in the nature of an amniocentesis procedure. Following aspiration of a desired volume of fluid, the needle can be removed. The puncture in the aneurysm sac may be patched, or left untreated provided the residual pressure in the aneurysm is sufficiently low.

A cannula may be inserted percutaneously into the patient's body, typically on the side of the patient's back, and advanced through the skin, muscle and other intervening tissues to a position where the distal end of the cannula is positioned within the isolated perigraft space, within the aneurysm. In applications where specific guidance of the cannula is desired to avoid damage to organs or critical anatomical structures, or for other reasons, the insertion and advancement of the cannula may be carried out under radiographic guidance or with the use of steriotaxis as known in the art, examples of such radiographic guidance and/or stereotaxis instruments and methods described in U.S. Pat. Nos. 4,733,661; 4,930,525 and 5,196,019, 5,053,042 and include those commercially available from various sources including the AccuPlace™ needle guide (In-Rad Corporation, Kentwood Mich.), the Bard CT Guide#550000 (C. R. Bard, Inc., Murray Hill, N.J.), the Picker Venue™ (Picker Corp., Cleveland, Ohio); and the Toshiba Aspire™ CT-fluoroscopy system (Toshiba America Medical Systems, Tustin, Calif.).

Alternatively, the cannula 20 may be inserted and advanced with the aid of electro-anatomical mapping and/or guidance devices and methods, examples of which are found in U.S. Pat. Nos. 5,647,361; 5,820,568; 5,730,128; 5,722,401; 5,578,007; 5,558,073; 5,465,717; 5,568,809; 5,694,945; 5,713,946; 5,729,129; 5,752,513; 5,833,608; 5,935,061; 5,931,818; 6,171,303; 5,931,818; 5,343,865; 5,425,370; 5,669,388; 6,015,414; 6,148,823 and 6,176,829 and are commercially available as the Carto™ or NOGA™ system available from Biosense-Webster, Inc., a Johnson & Johnson Company, Diamond Bar, Calif. and/or other systems available from Cardiac Pathways Corporation, 995 Benicia Avenue, Sunnyvale, Calif. and/or Stereotaxis, Inc., 4041 Forrest Park Avenue, St. Louis, Mo., or modifications thereof. See also U.S. Patent Application Publication No. 2003/0014075.

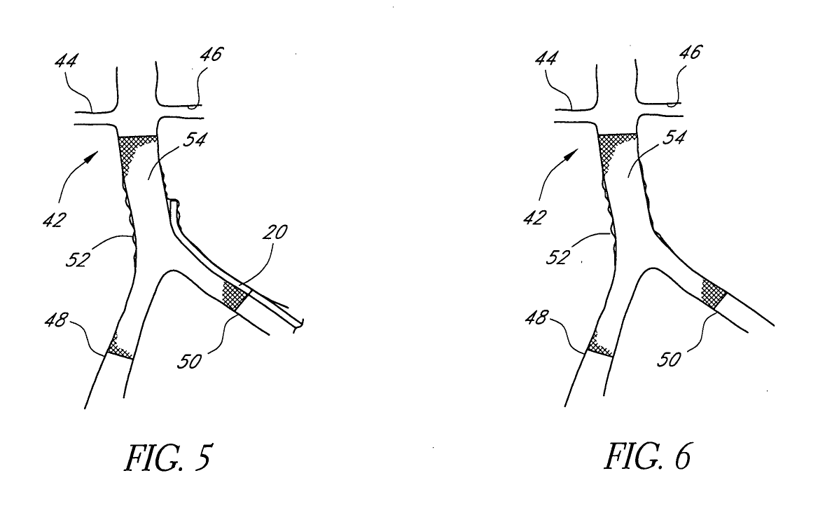

Returning to the illustrated embodiment, once the aneurysm has been isolated, the aspirating catheter 20 may be used to aspirate material from the aneurysm 52. As the aneurysm 52 is decompressed, the volume of the aneurysm sac is reduced as shown in FIG. 5. In this manner, the sac is pulled closer to the graft 54 as the volume of the sac is reduced.

The volume of blood removed may vary significantly from patient to patient, depending upon the configuration and size of the aneurysm and the desired clinical result. In general, sufficient blood is preferably aspirated to decompress the aneurysm and reduce the risk of rupture. Additional blood and debris may be removed to achieve a partial or complete regression of the aneurysm sac. Thus, the volume of the sac is reduced by at least 25% and often reduced by about 50%, and in certain applications reduced at least about 75% to about 90% of its original volume. Depending on the size of the aneurysm and the desired volume reduction, at least about 1 cc, often at least about 5 cc, and in certain applications at least about 10 cc or 15 cc or more of blood may be removed from the aneurysm. Contrast agent may be introduced into the aneurysm sac, to enhance visualization of the aneurysm sac volume during the aspiration step. A pressure sensor may also be provided on the catheter 20 to determine if the sac has been sufficiently aspirated. In one embodiment, the sac is aspirated until the pressure in the sac is between about 0 to about 40 millimeters of mercury and certain applications no more than about 20 millimeters of mercury. In one embodiment, the pressure sensor is positioned on the exterior of the catheter 20 near the distal end 24 and the distal access port 32. In certain embodiments, an anti-thrombolytic agent may be injected into the sac to dissolve blood clots in sac prior to aspiration.

The aspiration catheter 20 may also be used to deliver a medical agent into an isolated portion of the aneurysm 52. For example, in one embodiment, an embolization material may be delivered to the decompressed aneurysm sac 52 to further reduce the possibility of endoleaks. Decompression of the sac by removing a blood volume, prior to introduction of an embolization material or other agent, can minimize the pressure exerted on the sac by the introduction of an additional volume of material. The aspiration catheter 20 may then be removed leaving the graft 54 in place (see FIG. 6). Preferably, the graft 54 is a self-expandable graft such that graft expands to occupy the space vacated by the withdrawn catheter 20. In embodiments in which a medical agent is delivered, the catheter 20 may be a combined aspiration-injection catheter containing al least one aspiration lumen and at least one injection lumen. In other embodiments, the same lumen may be used for aspiration and injection.

With respect to embolization agents, in the prior art, it has also been difficult to estimate the amount (e.g., volume) of agent required to fill the sack. If too little agent is injected, endoleaks may not be sealed. If too much agent is injected into the sac, the pressure in the sack may rise causing the agent to spill into connecting vessels or the graft to be pushed away from the vessel wall. Accordingly, in one embodiment, the amount (e.g., volume) of aspirated blood is measured to determine the volume of the void to be filled with the embolization agent or other agent. An amount of embolization agent or other agent corresponding to the measured amount of aspirated blood is then injected into the sac. In general, the amount of embolization or other agent injected into the sac is generally less than the measured amount of aspirated blood such that the sac is not completely refilled thereby reducing the risk of rupturing the sac. Thus, less than about 90%, often less than about 75%, and in certain applications less than about 50% to 25% of the volume of the aspirated fluid in the form of an embolization agent is injected into the sac.

Depending upon the nature of the embolizing material, it may be compressed before deployment within an aneurysm. Once in contact with a bodily fluid, such as blood, the embolizing material may become saturated and expand. Embolizing material may have an open cellular structure, spongiform in nature, thereby increasing surface area and fluid saturation rate. The increased clotting surface coupled with enhanced blood saturation may provide means for accelerating thrombus formation. The open cellular structure may be produced by foaming methods known in art (e.g., foaming agents, salts, etc.). The nature of the embolizing material and foaming method may influence the compressibility and expansion characteristics of the material.

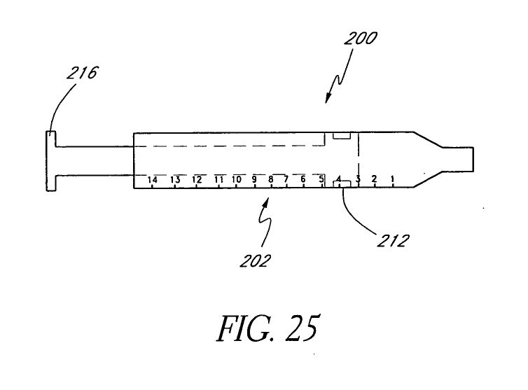

Accordingly, in determining the amount of agent corresponding to the measured amount of aspirated blood, consideration is preferably given to the expected expansion of the agent within the sac. For example, if the agent expands by about 100% then the injected amount of agent may be about 50% of the desired replacement volume. In one embodiment, the aspiration catheter 20 may be configured to deliver a calibrated amount of agent, which is a function of the amount of fluid aspirated from the sac. For example, those of skill in the art will recognize various arrangements, of syringes, pistons and the like that may be integrated into the catheter 20 and configured to deliver a pre-determined amount of agent that is based upon the amount of material removed from the sac. The pre-determined amount may also be based upon the desired replacement volume of the sac and/or the expected expansion of the agent within the sac. Such an arrangement may reduce user error. For example, FIG. 25 illustrates an embodiment of a proximal end of the aspiration catheter 20 described above. In this embodiment, the proximal end includes a syringe type member 200 for storing and injecting agent into the sac. The illustrated member 200 includes a syringe body 210 and a plunger 212 with a piston 214 at its distal end and a handle 216 at its proximal end. The member 200 may include a conversion scale 202 to indicate the amount of material to be inserted into the sac. In one embodiment, the scale 202 may be configured such that it takes into account the expected expansion of the agent. For example, in an embodiment in which the agent expands approximately 100%, the scale indicates the expected expanded volume of the agent instead of or in addition to the actual amount of agent injected (e.g., 2.5 cc of agent may be labeled 5 cc to indicate the expected expansion.). The scale 202 may also take into account the desire to not fill the sac completely as described above. For example, the scale 202 may be calibrated such that less than about 90%, often less than 75% and in certain applications less than about 50% to about 25% of the sac volume is filled with agent. Accordingly, in one embodiment, the surgeon may simply aspirate the sac noting the volume of the material removed. Using the member 200, the surgeon may inject an amount agent corresponding to the volume of material removed. The scale 202 may be configured to take into account the expected expansion and/or the desire to not entirely fill the sac. In this manner, the surgeon may focus on the amount of fluid removed from the sac and the chance for user error is reduced. The member 200 may be sold as part of a kit with the aspiration catheter 20.

In addition or in the alternative, a pressure sensor may be provided on the catheter used to deliver the agent. The pressure may be used as a guide for filling the sac. In one embodiment, the sac is filled until the pressure in the sac is less than about 40 millimeters of mercury and certain applications less than about 20 millimeters of mercury.

In yet another embodiment, the agent is delivered into the sac in multiple injections that are alternated with injections of a contrast agent. In such an embodiment, both the agent and the contrast agent can be injected into the sac through the same catheter 20 and/or through separate catheters (not shown). After and/or during injection of the contrast agent, a sacogram can be performed to visualize the extent to which the sac is filled and to which endoleaks are being prevented or reduced. It such embodiments it is generally preferred that with multiple injections the sac is filled from its distal end to its proximal end (distal and proximal with respect the graft as described herein). Thus, in between injections and/or during of the agent, the catheter 20 can be proximally withdrawn to fill the proximal portions of the sac. [0070] In one embodiment, embolizing material may be one or more hydrophilic foam materials such as polyurethane, polyvinyl alcohol, HYPAN® hydrogel, styrene/polyvinyl-pyrolodone (PVP) copolymer, polyacrylic acid copolymer, and the like. Such hydrophilic foam materials may provide superior mechanical strength compared to other hydrophilic foam gels. As a result, they may be more resistant to creep, migration, fracture, and other shortcomings. In another embodiment, embolizing material may be a hydrophobic foam material such as polyolefin, polyethylene, polypropylene, silicone, and vinyl acetate. Such hydrophobic materials are generally biocompatible and have been routinely used in the manufacture of endovascular devices. In another embodiment, the embolizing material may be expanded by a gas (e.g., carbon dioxide) to form a foam. For example, a two component bioglue comprising a protein (e.g., albumin) and a crosslinker (e.g., gluteraldehyde) may be used. The bioglue may be expanded when it is mixed with another component, which is the source for the gas (e.g., biocarbonate). As the components are mixed, the gas is released to form a foam. In one embodiment, the foam has an expansion ratio from about 2:1 to about 6:1. The foam preferably expands in less than about 30 seconds and often less than about 10 seconds and cures in less than about 5 minutes and often less than about 1 minute.

The embolizing material may include at least one therapeutic agent incorporated within and/or coated on its surface. The therapeutic agent may be a clotting factor (e.g., factors I-VIII, thrombin, fibrinogen), a tissue attachment factor (e.g., vitronectin, fibronectic, laminin, sclerosing agents: morrhuate sodium, ethanolamine oleate, tetradecyl sulfate), or other drug (e.g., anti-inflammation, antibiotics, etc.) . The clotting factors and the open cellular structure of the embolizing material may accelerate thrombus formation, after their release into the aneurysm. The thrombus may occlude the aneurysm from vascular blood flow thereby optimizing the healing response. The tissue attachment factors may promote the incorporation of the embolizing material within the vessel tissue thereby enhancing its retention. A radiopaque material may be incorporated in the embolizing material, for example, when it is being melted. The radiopaque material may include one or more of barium sulfate, gold, silver, tantalum oxide, tantalum, platinum, platinum/iridium alloy, tungsten, and other materials used for imaging purposes.

The embolizing material may be thermoplastic thereby allowing melting and reshaping by extrusion, casting, thermal forming, and like processes. Embolizing material may be shaped and sized in a variety of geometries such as pellets, spheres, non-uniform shapes, or cylinders, as shown. The appropriate embolizing material shape and size may be determined by application and achieved by one of skill in the art.

The expansile polymeric material may comprise a hydrogel. Preferable hydrogels include a biocompatible, macroporous, hydrophilic hydrogel foam material as described in U.S. Pat. No. 5,570,585 (Park et al.), the entirety of which is expressly incorporated herein by reference as well as other hydrogels that undergo controlled volumetric expansion in response to changes in such environmental parameters as pH or temperature. An example of one such hydrogel that undergoes controlled volumetric expansion in response to changes in is environment is described in U.S. patent application Ser. No. 09/867,340, the entirety of which is expressly incorporated herein by reference. These pH responsive hydrogels are prepared by forming a liquid mixture that contains (a) at least one monomer and/or polymer, at least a portion of which is sensitive to changes in an environmental parameter; (b) a cross-linking agent; and (c) a polymerization initiator. If desired, a porosigen (e.g., NaCl, ice crystals, or sucrose) may be added to the mixture, and then removed from the resultant solid hydrogel to provide a hydrogel with sufficient porosity to permit cellular ingrowth. The controlled rate of expansion is provided through the incorporation of ethylenically unsaturated monomers with ionizable functional groups (e.g., amines, carboxylic acids). For example, if acrylic acid is incorporated into the crosslinked network, the hydrogel is incubated in a low pH solution to protonate the carboxylic acids. After the excess low pH solution is rinsed away and the hydrogel dried, the hydrogel can be introduced through a microcatheter filled with saline at physiological pH or with blood. The hydrogel cannot expand until the carboxylic acid grous deprotonate. Conversely, if an amine containing monomer is incorporated into the crosslinked network, the hydrogel is incubated in a high pH solution to deprotonate amines. After the excess high pH solution is rinsed away and the hydrogel dried, the hydrogel can be introduced through a microcatheter filled with saline at physiological pH or with blood. The hydrogel cannot expand until the amine groups protonate.

In one formulation of the hydrogel, the monomer solution is comprised of ethylenically unsaturated monomers, an ethylenically unsaturated crosslinking agent, a porosigen, and a solvent. At least a portion, from about 10% to about 50%, and preferably about 10% to about 30%, of the monomers selected is pH sensitive. The preferred pH sensitive monomer is acrylic acid. Methacrylic acid and derivatives of both acids will also impart pH sensitivity. Since the mechanical properties of hydrogels prepared exclusively with these acids are poor, a monomer to provide additional mechanical properties should be selected. A preferred monomer for providing mechanical properties is acrylamide, which may be used in combination with one or more of the above-mentioned pH sensitive monomers to impart additional compressive strength or other mechanical properties. Preferred concentrations of the monomers m the solvent range from 20% w/w to 30% w/w.

The crosslinking agent can be any of a variety of multifunctional ethylenically unsaturated compounds, preferably N,N′-methylenebisacrylamide. If biodegradation of the hydrogel material is desired, a biodegradable crosslinking agent should be selected. The concentrations of the crosslinking agent in the solvent should be less than about 1% w/w, and preferably less than about 0.1% w/w.

The porosity of the hydrogel material is provided by a supersaturated suspension of a porosigen in the monomer solution. A porosigen that is not soluble in the monomer solution, but is soluble in the washing solution can also be used. Sodium chloride is the preferred porosigen, but potassium chloride, ice, sucrose, and sodium bicarbonate can also be used. It is preferred to control the particle size of the porosigen to less than about 25 microns, more preferably less than about 10 microns. The small particle size aids in the suspension of the porosigen in the solvent. Preferred concentrations of the porosigen range from about 5% w/w to about 50% w/w, more preferably about 10% w/w to about 20% w/w, in the monomer solution. Alternatively, the porosigen can be omitted and a non-porous hydrogel can be fabricated.

The solvent, if necessary, is selected based on the solubilities of the monomers, crosslinking agent, and porosigen. If a liquid monomer (e.g. 2hydroxyethyl methacrylate) is used, a solvent is not necessary. A preferred solvent is water, but ethyl alcohol can also be used. Preferred concentrations of the solvent range from about 20% w/w to about 80% w/w, more preferably about 50% w/w to about 80% w/w.

The crosslink density substantially affects the mechanical properties of these hydrogel materials. The crosslink density (and hence the mechanical properties) can best be manipulated through changes in the monomer concentration, crosslinking agent concentration, and solvent concentration. The crosslinking of the monomer can be achieved through reduction-oxidation, radiation, and heat. Radiation crosslinking of the monomer solution can be achieved with ultraviolet light and visible light with suitable initiators or ionizing radiation (e.g. electron beam or gamma ray) without initiators. A preferred type of crosslinking initiator is one that acts via reduction-oxidation. Specific examples of such red/ox initiators include ammonium persulfate and N,N,N′,N′-tetrarnethylethylenediamine.

After the polymerization is complete, the hydrogen is washed with water, alcohol or other suitable washing solution(s) to remove the porosigen(s), any unreacted, residual monomer(s) and any unincorporated oligomers. Preferably this is accomplished by initially washing the hydrogel in distilled water.

As discussed above, the control of the expansion rate of the hydrogel is achieved by protonation/deprotonaton of the ionizable functional groups present on the hydrogel network. Once the hydrogel has been prepared and the excess monomer and porosigen have been washed away, the steps to control the rate of expansion can be performed.

In formulations where pH sensitive monomers with carboxylic acid groups have been incorporated into the hydrogel network, the hydrogel is incubated in a low pH solution. The free protons in the solution protonate the carboxylic acid groups on the hydrogel network. The duration and temperature of the incubation and the pH of the solution influence the amount of control on the expansion rate. Generally, the duration and temperature of the incubation are directly proportional to the amount of expansion control, while the solution pH is inversely proportional. The water content of the treating solution may also affect the expansion control. In this regard, the hydrogel is able to expand more in the treating solution and it is presumed that an increased number of carboxylic acid groups are available for protonation. An optimization of water content and pH is required for maximum control on the expansion rate. After the incubation is concluded, the excess treating solution is washed away and the hydrogel material is dried. The hydrogel treated with the low pH solution may dry down to a smaller dimension than the untreated hydrogel.

In formulations where pH sensitive monomers with amine groups were incorporated into the hydrogel network, the hydrogel is incubated in high pH solution. Deprotonation then occurs on the amine groups of the hydrogel network at high pH. The duration and temperature of the incubation, and the pH of the solution, influence the amount of control on the expansion rate. Generally, the duration, temperature, and solution pH of the incubation are directly proportional to the amount of expansion control. After the incubation is concluded, the excess treating solution is washed away and the hydrogel material is dried.

Examples of other biodegradable, expansile hydrogels include, but are not necessarily limited to those described in U.S. Pat. Nos. 5,162,430 (Rhee et al.), 5,410,016 (Hubbell et al.), 5,990,237 (Bentley et al.), 6,177,095 (Sawhney et al.), 6,184,266 B1 (Ronan et al.), 6,201,065 B1 (Pathak et al.), 6,224,892 B1 (Searle), 5,980,550 (Eder et al.) and PCT International Patent Publication Nos. WO 00/44306 (Murayama et al.), WO 00/74577 (Wallace et al.).

The expansile polymeric material, whether a hydrogel or other type of polymer, may be mixed with a carrier fluid to facilitate delivery into the body. In cases where the expansile polymeric material is in the form of solid pellets or particles, those pellets or particles may be suspended in a liquid carrier, such as saline, polyethylene glycol or a radiographic contrast medium. Alternatively, one or more solid pieces of the expansible polymeric material me be formed, mounted on or attached to a carrier member to facilitate introduction of the polymeric material into the aneurysm sac. See also U.S. Patent Application Publication No. 2003/0204246.

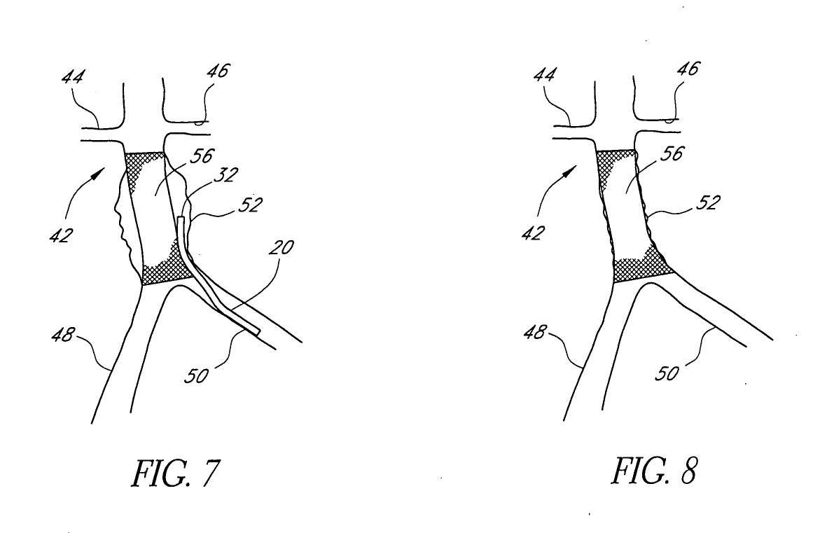

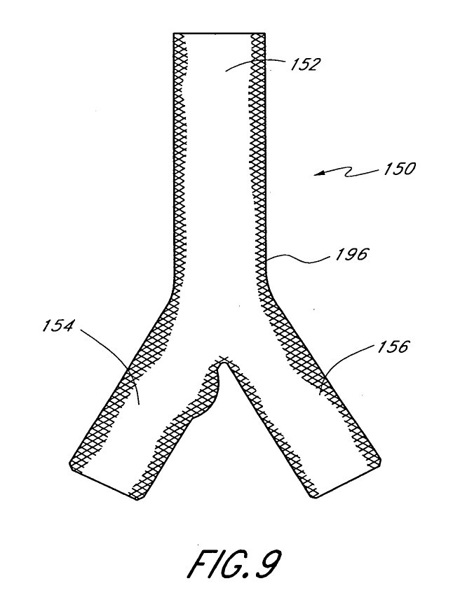

With reference to FIGS. 7 and 8, a straight graft 56 has been deployed to isolate the abdominal aortic aneurysm. In addition, the distal access port 32 of the aspiration lumen is position such that it is in communication with in the aneurysm. As with the previous embodiment, with the aneurysm isolated, the aspirating catheter 20 may be used to aspirate material from the aneurysm to reduce the volume of the aneurysm sac as shown in FIG. 8. In this manner, the sac is pulled closer to the graft 54 as the volume of the sac is reduced. As mentioned above, before or after aspiration, the aspiration catheter 20 may also be used to deliver a medical agent (e.g., embolization material) to the sac to further reduce the possibility of endoleaks. The aspiration catheter 20 may then be removed leaving the graft 54 in place.

The techniques and methods described above are particularly useful in reducing endoleaks in an abdominal aortic aneurysm sac that has been isolated by a graft. However, those of skill in the art will recognize that these methods may also be adapted to other surgical applications. For example, the aspiration catheter 20 may be used to aspirate thoracic aneurysms or an aneurysm in the neurovascular system (e.g., a Berry aneurysm) that has been isolated with a graft.

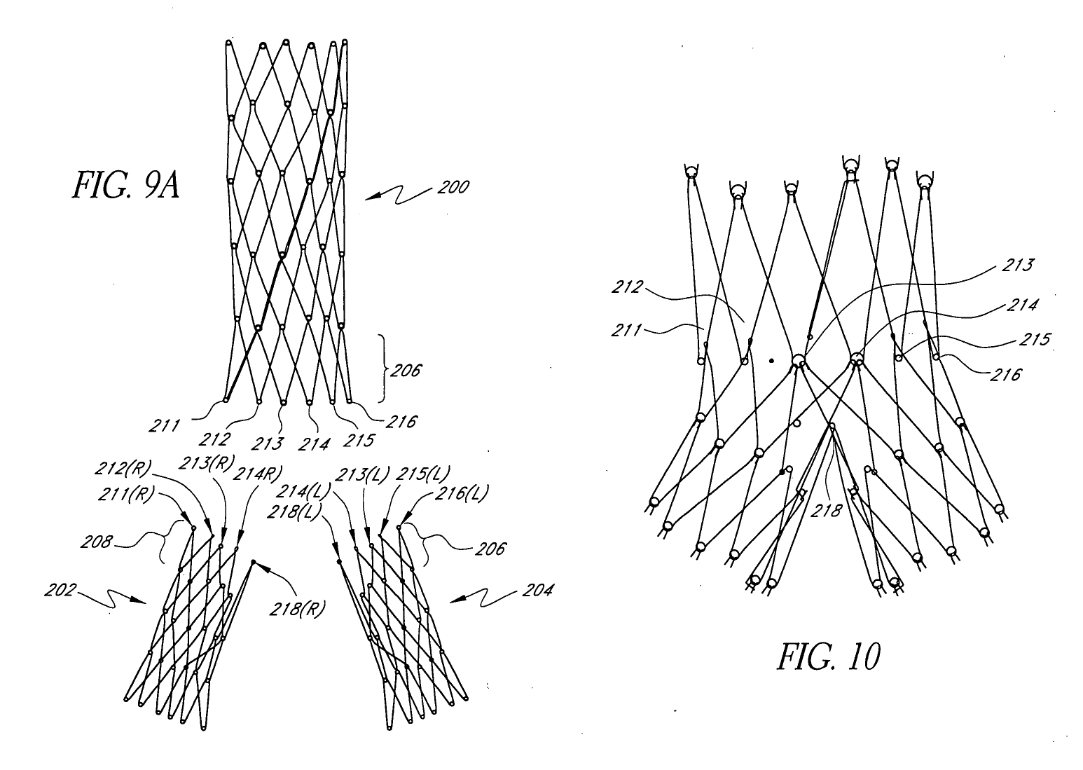

With reference to FIG. 9, there is disclosed a schematic representation of an exemplary bifurcated graft 150 that comprises a main body 152, an ipsilateral iliac branch 154 and a contralateral iliac branch 156. FIG. 9A is an exploded schematic representation of an exemplary a hinged or articulated tubular wire support structure for self-expanding the graft 150 following placement to isolate an abdominal aortic aneurysm as described above. Additional embodiments and further details of the exemplary embodiment can be found in (i) U.S. Pat. No. 6,197,049, entitled “ENDOLUMINAL VASCULAR GRAFT”, (ii) U.S. patent application Ser. No. 09/891,620, filed Jun. 26, 2001, entitled “IMPLANTABLE VASCULAR GRAFT” and published under U.S. Publication No. 2002-0052644A1 and (iii) PCT Publication WO0239888A2, entitled “IMPLANTABLE VASCULAR GRAFT”, the disclosures of which are incorporated in their entirety herein by reference.

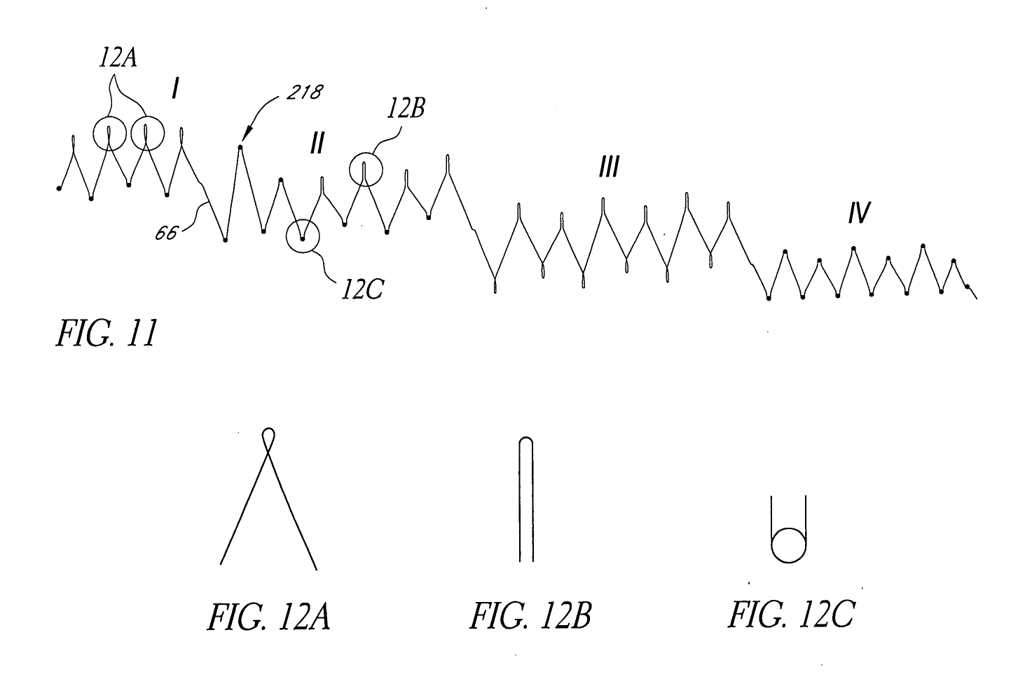

The tubular wire support comprises a main body, or aortic trunk portion 200 and right 202 and left 204 iliac branch portions. Right and left designations correspond to the anatomic designations of right and left common iliac arteries. The proximal end 206 of the aortic trunk portion 200 has apexes 211-216 adapted for connection with the complementary apexes on the distal ends 208 and 210 of the right 202 and left 204 iliac branch portions, respectively. Complementary pairing of apexes is indicated by the shared numbers, wherein the right branch portion apexes are designated by (R) and the left branch portion apexes are designated by (L). Each of the portions may be formed from a continuous single length of wire. See FIG. 11.

With reference to FIG. 10, the assembled articulated wire support structure 199 is shown. The central or medial apex 213 in the foreground (anterior) of the aortic trunk portion 200 is linked with 213(R) on the right iliac portion 202 and 213(L) on the left iliac portion 204. Similarly, the central apex 214 in the background (posterior) is linked with 214(R) on the right iliac portion 202 and 214(L) on the left iliac portion 204. Each of these linkages has two iliac apexes joined with one aortic branch apex. The linkage configurations may be of any of the variety described above in FIGS. 7A-D. The medial most apexes 218 (R) and (L) of the iliac branch portions 202 and 204 are linked together, without direct connection with the aortic truck portion 200.

The medial apexes 213 and 214 function as pivot points about which the right and left iliac branches 202, 204 can pivot to accommodate unique anatomies. Although the right and left iliac branches 202, 204 are illustrated at an angle of about 45 degrees to each other, they are articulable through at least an angle of about 90 degrees and preferably at least about 120 degrees. The illustrated embodiment allows articulation through about 180 degrees while maintaining patency of the central lumen. To further improve patency at high iliac angles, the apexes 213 and 214 can be displaced proximally from the transverse plane which roughly contains apexes 211, 212, 215 and 216 by a minor adjustment to the fixture about which the wire is formed. Advancing the pivot point proximally relative to the lateral apexes (e.g., 211, 216) opens the unbiased angle between the iliac branches 202 and 204.

In the illustrated embodiment, the pivot point is formed by a moveable link between an eye on apex 213 and two apexes 213R and 213L folded therethrough. To accommodate the two iliac apexes 213R and 213L, the diameter of the eye at apex 213 may be slightly larger than the diameter of the eye on other apexes throughout the graft. Thus, for example, the diameter of the eye at apex 213 in one embodiment made from 0.014″ diameter wire is about 0.059″, compared to a diameter of about 0.020″ for eyes elsewhere in the graft.

Although the pivot points (apexes 213, 214) in the illustrated embodiment are on the medial plane, they may be moved laterally such as, for example, to the axis of each of the iliac branches. In this variation, each iliac branch will have an anterior and a posterior pivot link on or about its longitudinal axis, for a total of four unique pivot links at the bifurcation. Alternatively, the pivot points can be moved as far as to lateral apexes 211 and 216. Other variations will be apparent to those of skill in the art in view of the disclosure herein.

To facilitate lateral rotation of the iliac branches 202, 204 about the pivot points and away from the longitudinal axis of the aorta trunk portion 200 of the graft, the remaining links between the aorta trunk portion 200 and the iliac branches 202, 204 preferably permit axial compression and expansion. In general, at least one and preferably. several links lateral to the pivot point in the illustrated embodiment permit axial compression or shortening of the graft to accommodate lateral pivoting of the iliac branch. If the pivot point is moved laterally from the longitudinal axis of the aorta portion of the graft, any links medial of the pivot point preferably permit axial elongation to accommodate lateral rotation of the branch. In this manner, the desired range of rotation of the iliac branches may be accomplished with minimal deformation of the wire, and with patency of the graft optimized throughout the angular range of motion.

To permit axial compression substantially without deformation of the wire, the lateral linkages, 211 and 212 for the right iliac, and 215 and 216 for the left iliac, may be different from the apex-to-apex linkage configurations illustrated elsewhere on the graft. The lateral linkages are preferably slidable linkages, wherein a loop formed at the distal end of the iliac apex slidably engages a strut of the corresponding aortic truck portion. The loop and strut orientation may be reversed, as will be apparent to those of skill in the art. Interlocking “elbows” without any distinct loop may also be used. Such an axially compressible linkage on the lateral margins of the assembled wire support structure allow the iliac branch portions much greater lateral flexibility, thereby facilitating placement in patients who often exhibit a variety of iliac branch asymmetries and different angles of divergence from the aortic trunk.

Referring to FIG. 11, there is illustrated a plan view of a single formed wire used for rolling about a longitudinal axis to produce a four segment straight tubular wire support for an iliac limb. The formed wire exhibits distinct segments, each corresponding to an individual tubular segment in the tubular supports 202 or 204 (See FIG. 9). The distal segment I, is adapted to articulate with the aortic trunk portion 200 and the adjacent iliac limb portion. The distal segment (I) has two apexes (e.g. corresponding to 211 and 212 on the right iliac portion 202 in FIG. 9) which form a loop adapted to slidably engage a strut in the lateral wall of the aortic portion. These articulating loops (A) are enlarged in FIG. 12A. As discussed above, the loops are preferably looped around a strut on the corresponding apex of the proximal aortic segment to provide a sliding linkage.

The apex 218 is proximally displaced relative to the other four apexes in the distal segment (I). Apex 218 (R or L) is designed to link with the complementary 218 apex on the other iliac branch portion (See FIG. 10). The apex 218 in the illustrated embodiment is formed adjacent or near an intersegment connector 66, which extends proximally from the distal segment.

The other apexes on the distal segment (I) of an iliac limb are designed to link with a loop on the corresponding apex of the proximal aortic segment. Because many variations of this linkage are consistent with the present invention (See U.S. Pat. No. 6,197,049, issued Mar. 6, 200, entitled “ARTICULATED BIFURCATION GRAFT”, the disclosure of which was incorporated above), the form of the corresponding apexes may vary. In a preferred variation, the apexes (B) form a narrow U-shape, having an inside diameter of about 0.019″ in an embodiment made from 0.012″ Conichrome wire (tensile strength 300 ksi minimum) as illustrated in FIG. 12B. The U-shaped, elongated axial portion of the apex shown in FIG. 12B permits the apex to be wrapped through and around a corresponding loop apex of the proximal aortic segment.

In more general terms, the wire support illustrated in FIGS. 9A and 10 comprises a main body support structure formed from one or more lengths of wire and having a proximal end, a distal end and a central lumen extending along a longitudinal axis. The wire support also comprises a first branch support structure formed from one or more lengths of wire and having a proximal end, a distal end and a central lumen therethrough. The first branch support structure is pivotably connected to the proximal end of the main body support structure. The tubular wire support further comprises a second branch support structure formed from one or more lengths of wire and having a proximal end, a distal end and a central lumen extending therethrough. The distal end of the second branch support structure is pivotably connected to the proximal end of the main body support structure.

Further, the distal ends of the first and second branch structures may be joined together by a flexible linkage, formed for example between apexes 218(R) and 218(L) in FIG. 9A. By incorporating a medial linkage between the two branch support structures and pivotable linkages with the main trunk, the first and second branch support structures can hinge laterally outward from the longitudinal axis without compromising the volume of the lumen. Thus, the branches may enjoy a wide range of lateral movement, thereby accommodating a variety of patient and vessel heterogeneity. Additional corresponding apexes between the main trunk and each iliac branch may also be connected, or may be free floating within the outer polymeric sleeve. Axially compressible lateral linkages, discussed above and illustrated in FIG. 10, may optionally be added.

The proximal apexes (C) of the iliac limb portions are adapted to link with the distal apexes of the next segment. These proximal apexes preferably form loops, such as those illustrated in FIG. 12C, wherein the elongated axial portions of the corresponding proximal apex in the adjacent segment can wrap around the loop, thereby providing flexibility of the graft.

The wire may be made from any of a variety of different alloys and wire diameters or non-round cross-sections, as has been discussed. In one embodiment of the bifurcation graft, the wire gauge remains substantially constant throughout the aorta component and steps down to a second, smaller cross-section throughout the iliac component.

A wire diameter of approximately 0.018″ may be useful in the aorta trunk portion of a graft having five segments each having 2.0 cm length per segment, each segment having six struts intended for use in the aorta, while a smaller diameter such as 0.012″ might be useful for segments of the graft having 6 struts per segment intended for the iliac artery.

In one embodiment, the wire diameter may be tapered throughout from the proximal to distal ends of the aorta section and/or iliac section. Alternatively, the wire diameter may be tapered incremental or stepped down, or stepped up, depending on the radial strength requirements of each particular clinical application. In one embodiment, intended for the abdominal aortic artery, the wire has a cross-section of about 0.018″ in the proximal zone and the wire tapers down regularly or in one or more steps to a diameter of about 0.012″ in the distal zone of the graft. End point dimensions and rates of taper can be varied widely, within the spirit of the present invention, depending upon the desired clinical performance.

In general, in the tapered or stepped wire embodiments, the diameter of the wire in the iliac branches is no more than about 80% of the diameter of the wire in the aortic trunk. This permits increased flexibility of the graft in the region of the iliac branches, which has been determined by the present inventors to be clinically desirable.

The collapsed prosthesis in accordance with this embodiment has a diameter in the range of about 2 to about 10 mm. Preferably, the maximum diameter of the collapsed prosthesis is in the range of about 3 to 6 mm (12 to 18 French). Some embodiments of the delivery catheter including the prosthesis will be in the range of from 18 to 20 or 21 French; other embodiments will be as low as 19 F, 16 F, 14 F, or smaller. After deployment, the expanded endolumenal vascular prosthesis has radially self-expanded to a diameter anywhere in the range of about 20 to 40 mm, corresponding to expansion ratios of about 1:2 to 1:20. In a preferred embodiment, the expansion ratios range from about 1:4 to 1:8, more preferably from about 1:4 to 1:6.

The wire may be made from any of a variety of different materials, such as elgiloy, Nitinol or MP35N, or other alloys which include nickel, titanium, tantalum, or stainless steel, high Co—Cr alloys or other temperature sensitive materials. For example, an alloy comprising Ni 15%, Co 40%, Cr 20%, Mo 7% and balance Fe may be used. The tensile strength of suitable wire is generally above about 300 Ksi and often between about 300 and about 340 Ksi for many embodiments. In one embodiment, a Chromium-Nickel-Molybdenum alloy such as that marketed under the name Conichrom (Fort Wayne Metals, Ind.) has a tensile strength ranging from 300 to 320 K psi, elongation of 3.5-4.0%. The wire may be treated with a plasma coating and be provided with or without additional coatings such as PTFE, Teflon, Perlyne and drugs.

Although the above embodiments have been described primarily in the context of formed wire, the support structure may conveniently be formed from a flat sheet or tube of material such as Elgiloy, Nitinol, or other material having desired physical properties. Sheets having a thickness of no more than about 0.025″ and, preferably, no more than about 0.015″ are useful for this purpose. In one embodiment, the support structure is formed by laser cutting the appropriate pattern on a 0.014″ thickness Elgiloy foil or tube. Similarly, any of the other embodiments disclosed previously herein can be manufactured by laser cutting, chemical etching, or otherwise forming the wire cage support from a flat sheet or tube of Elgiloy or other suitable material.

The endolumenal prosthesis 150 illustrated and described above depicts an embodiment in which the polymeric sleeve 196 (see FIG. 9) may be situated concentrically outside of the tubular wire support. However, other embodiments may include a sleeve or sleeves situated substantially concentrically inside the wire support, or on both the inside and the outside of the wire support. Alternatively, the wire support may be embedded within a polymeric matrix which makes up the sleeve. Regardless of whether the sleeve is inside or outside the wire support, or both inside and outside, the sleeve may be attached to the wire support by any of a variety of methods or devices, including laser bonding, adhesives, clips, sutures, dipping or spraying or others, depending upon the composition of the sleeve or membrane and overall graft design.

The sleeve or membrane that is used to cover the tubular wire graft cage can be manufactured from any of a variety of synthetic polymeric materials, or combinations thereof, including DACRON®, polyester, polyethylene, polypropylene, fluoropolymers, polyurethane foamed films, silicon, nylon, silk, thin sheets of super-elastic materials, woven materials, polyethylene terephthalate (PET), or any other biocompatible material. In one embodiment of the present invention, the membrane material is a fluoropolymer, in particular, expanded polytetrafluoroethylene (ePTFE) having a node-fibril structure. The ePTFE membrane used in the present invention is manufactured from thin films of ePTFE that are each approximately 0.0025 to 0.025 mm in thickness. Thus, the films could be 0.0025, 0.0050, 0.0075, 0.0100, 0.0125, 0.0150, 0.0175, 0.0200, 0.0225, and 0.0250 mm thick.

From 1 to about 200 plies (layers) of ePTFE film may be stacked up and laminated to one another to obtain a membrane with the desired mechanical and structural properties. An even number of layers are preferably stacked together (e.g., 2, 4, 6, 8, 10, etc.), with approximately 2 to 20 layers being desirable. Cross-lamination occurs by placing superimposed sheets on one another such that the film drawing direction, or stretching direction, of each sheet is angularly offset by angles between 0 degrees and 180 degrees from adjacent layers or plies. Because the base ePTFE is thin, as thin as 0.0025 mm thick, superimposed films can be rotated relative to one another to improve mechanical properties of the membrane. In one preferred embodiment, the membrane is manufactured by laminating between 4 to 8 plies of ePTFE film, each film ply being about 0.0125 mm thick.

Additional details and modified embodiments of the graft the polymeric sleeve may be found in co-pending U.S. patent application Ser. No. 10/820,455, entitled “Endoluminal Vascular Prosthesis With Neointima Inhibiting EPTFE Polymeric Sleeve”, filed Apr. 8, 2004, the disclosure of which is hereby incorporated herein by reference in its entirety.

The self expandable bifurcation graft of the exemplary embodiment described above can be deployed at a treatment site in accordance with any of a variety of techniques as will be apparent to those of skill in the art. One such technique is disclosed in U.S. Pat. No. 6,090,128, entitled “Bifurcated Vascular Graft Deployment Device” and issued Jul. 7, 2000, the disclosure of which is incorporated in its entirety herein by reference. Other techniques are disclosed in U.S. Pat. No. 6,261,316, entitled “Single Puncture Bifurcation Graft Deployment System”, the disclosure of which is incorporated in its entirety herein by reference.

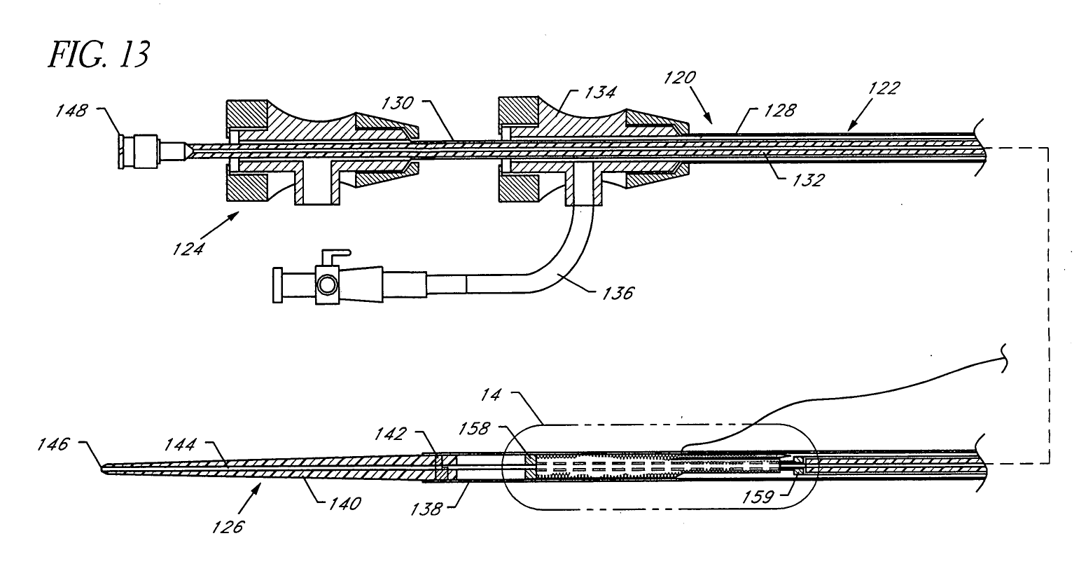

A partial cross-sectional side elevational view of one deployment apparatus 120 in accordance with one embodiment is shown in FIG. 13. Additional embodiments and further details of this deployment apparatus 120 are disclosed in U.S. Pat. No. 6,660,030, issued Dec. 9, 2003, entitled “Bifurcation Graft Deployment Catheter”, the disclosure of which is incorporated in its entirety herein by reference. In this particular embodiment, the deployment apparatus 120 comprises an elongate flexible multicomponent tubular body 122 having a proximal end 124 and a distal end 126. The tubular body 122 and other components of this system can be manufactured in accordance with any of a variety of techniques well known in the catheter manufacturing field. Suitable materials and dimensions can be readily selected taking into account the natural anatomical dimensions in the iliacs and aorta, together with the dimensions of the desired percutaneous access site.

The elongate flexible tubular body 122 comprises an outer sheath 128 which is axially movably positioned upon an intermediate tube 130. A central tubular core 132 is axially movably positioned within the intermediate tube 130. In one embodiment, the outer tubular sheath comprises extruded PTFE, having an outside diameter of about 0.250″ and an inside diameter of about 0.230″. The tubular sheath 128 is provided at its proximal end with a manifold 134, having a hemostatic valve 136 thereon and access ports such as for the infusion of drugs or contrast media as will be understood by those of skill in the art.

The outer tubular sheath 128 has an axial length within the range of from about 30″ to about 40″, and, in one embodiment of the deployment device 120 having an overall length of 105 cm, the axial length of the outer tubular sheath 128 is about 46 cm and the outside diameter is no more than about 0.250″. Thus, the distal end of the tubular sheath 128 is located at least about 16 cm proximally of the distal end 126 of the deployment catheter 120 in stent loaded configuration.

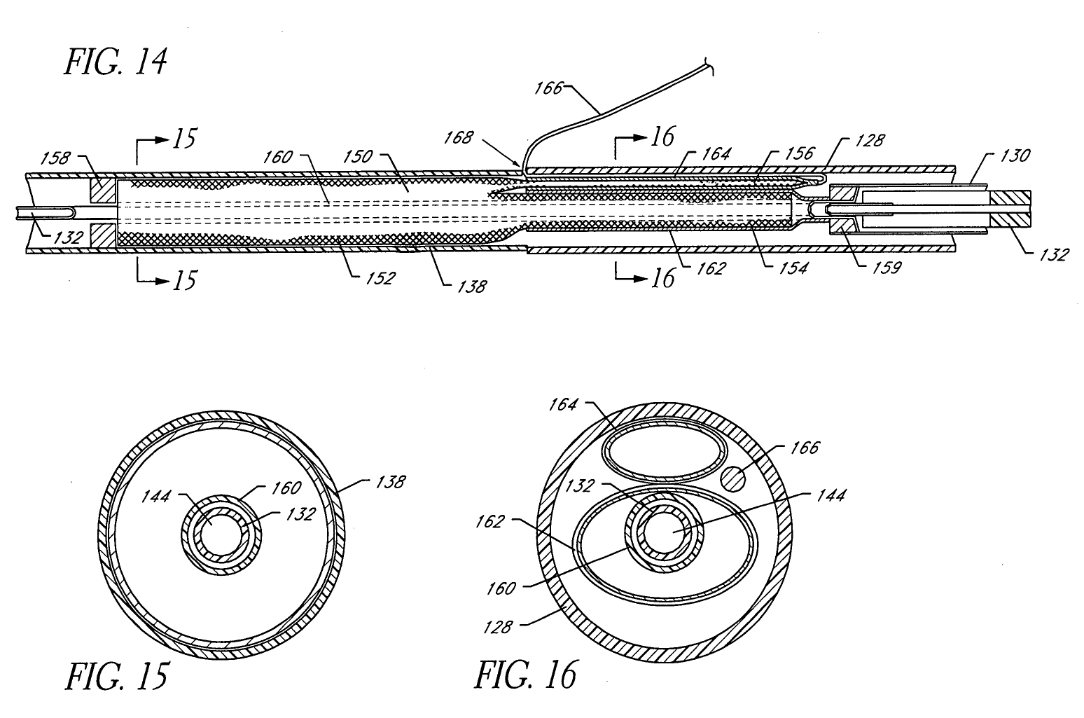

As can be seen from FIGS. 14-16, proximal retraction of the outer sheath 128 with respect to the intermediate tube 130 will expose the compressed iliac branches of the graft, as will be discussed in more detail below.

A distal segment of the deployment catheter 120 comprises an outer tubular housing 138, which terminates distally in an elongate flexible tapered distal tip 140. The distal housing 138 and tip 140 are axially immovably connected to the central core 132 at a connection 142.

The distal tip 140 preferably tapers from an outside diameter of about 0.225″ at its proximal end to an outside diameter of about 0.070″ at the distal end thereof. The overall length of the distal tip 140 in one embodiment of the deployment catheter 120 is about 3″. However, the length and rate of taper of the distal tip 140 can be varied depending upon the desired trackability and flexibility characteristics. The distal end of the housing 138 is secured to the proximal end of the distal tip 140 such as by thermal bonding, adhesive bonding, and/or any of a variety of other securing techniques known in the art. The proximal end of distal tip 140 is preferably also directly or indirectly connected to the central core 132 such as by a friction fit and/or adhesive bonding.

In at least the distal section of the catheter, the central core 132 preferably comprises a length of hypodermic needle tubing. The hypodermic needle tubing may extend throughout the length catheter to the proximal end thereof, or may be secured to the distal end of a proximal extrusion. A central guidewire lumen 144 extends throughout the length of the tubular central core 132, having a distal exit port 146 and a proximal access port 148 as will be understood by those of skill in the art.

Referring to FIGS. 14-16, the bifurcated endolumenal graft 150 is illustrated in a compressed configuration within the deployment catheter 120. As mentioned above, the graft 150 comprises a distal aortic section or main body 152, a proximal ipsilateral iliac portion 154, and a proximal contralateral iliac portion 156. The aortic trunk portion 152 of the graft 150 is contained within the tubular housing 138. Distal axial advancement of the central tubular core 132 will cause the distal tip 140 and housing 138 to advance distally with respect to the graft 150, thereby permitting the aortic trunk portion 152 of the graft 150 to expand to its larger, unconstrained diameter. Distal travel of the graft 150 is prevented by a distal stop 158 which is axially immovably connected to the intermediate tube 130. Distal stop 158 may comprise any of a variety of structures, such as an annular flange or component which is adhered to, bonded to or integrally formed with a tubular extension 160 of the intermediate tube 132. Tubular extension 160 is axially movably positioned over the hypotube central core 132.

The tubular extension 160 extends axially throughout the length of the graft 150. At the proximal end of the graft 150, a step 159 axially immovably connects the tubular extension 160 to the intermediate tube 130. In addition, the step 159 provides a proximal stop surface to prevent proximal travel of the graft 150 on the catheter 120. The function of step 159 can be accomplished through any of a variety of structures as will be apparent to those of skill in the art in view of the disclosure herein. For example, the step 159 may comprise an annular ring or spacer which receives the tubular extension 160 at a central aperture therethrough, and fits within the distal end of the intermediate tube 130. Alternatively, the intermediate tube 130 can be reduced in diameter through a generally conical section or shoulder to the diameter of tubular extension 160.

Proximal retraction of the outer sheath 128 will release the iliac branches 154 and 156 of the graft 150. The iliac branches 154 and 156 will remain compressed, within a first (ipsilateral) tubular sheath 162 and a second (contralateral) tubular sheath 164. The first tubular sheath 162 is configured to restrain the ipsilateral branch of the graft 150 in the constrained configuration, for implantation at the treatment site. The first tubular sheath 162 is adapted to be axially proximally removed from the iliac branch, thereby permitting the branch to expand to its implanted configuration. In one embodiment, the first tubular sheath 162 comprises a thin walled PTFE extrusion having an outside diameter of about 0.215″ and an axial length of about 7.5 cm. A proximal end of the tubular sheath 162 is necked down such as by heat shrinking to secure the first tubular sheath 162 to the tubular extension 160. In this manner, proximal withdrawal of the intermediate tube 130 will in turn proximally advance the first tubular sheath 162 relative to the graft 150, thereby deploying the self expandable iliac branch of the graft 150.

The second tubular sheath 164 is secured to the contralateral guidewire 166 (equivalent to guidewire 66 discussed previously), which extends outside of the tubular body 122 at a point 168, such as may be conveniently provided at the junction between the outer tubular sheath 128 and the distal housing 138. The second tubular sheath 164 is adapted to restrain the contralateral branch of the graft 150 in the reduced profile. In one embodiment of the invention, the second tubular sheath 164 has an outside diameter of about 0.215″ and an axial length of about 7.5 cm. The second tubular sheath 164 can have a significantly smaller cross-section than the first tubular sheath 162, due to the presence of the tubular core 132 and intermediate tube 130 within the first iliac branch 154.

The second tubular sheath 164 is secured at its proximal end to a distal end of the contralateral guidewire 166. This may be accomplished through any of a variety of securing techniques, such as heat shrinking, adhesives, mechanical interfit and the like. In one embodiment, the guidewire is provided with a knot or other diameter enlarging structure to provide an interference fit with the proximal end of the second tubular sheath 156, and the proximal end of the second tubular sheath 156 is heat shrunk and/or bonded in the area of the knot to provide a secure connection. Any of a variety of other techniques for providing a secure connection between the contralateral guidewire 166 and tubular sheath 156 can readily be used in the context of the present invention as will be apparent to those of skill in the art in view of the disclosure herein. The contralateral guidewire 166 can comprise any of a variety of structures, including polymeric monofilament materials, braided or woven materials, metal ribbon or wire, or conventional guidewires as are well known in the art.

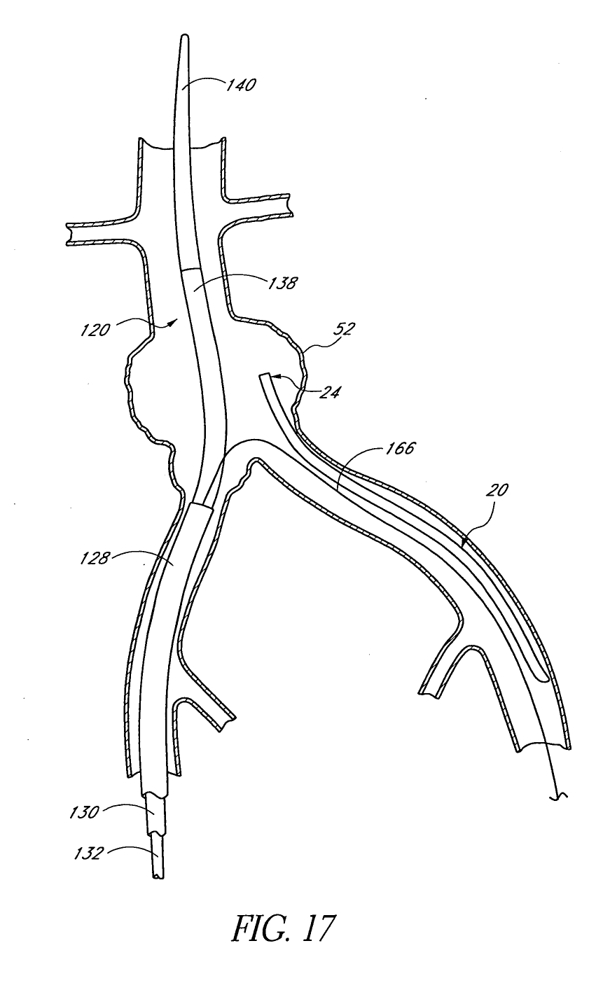

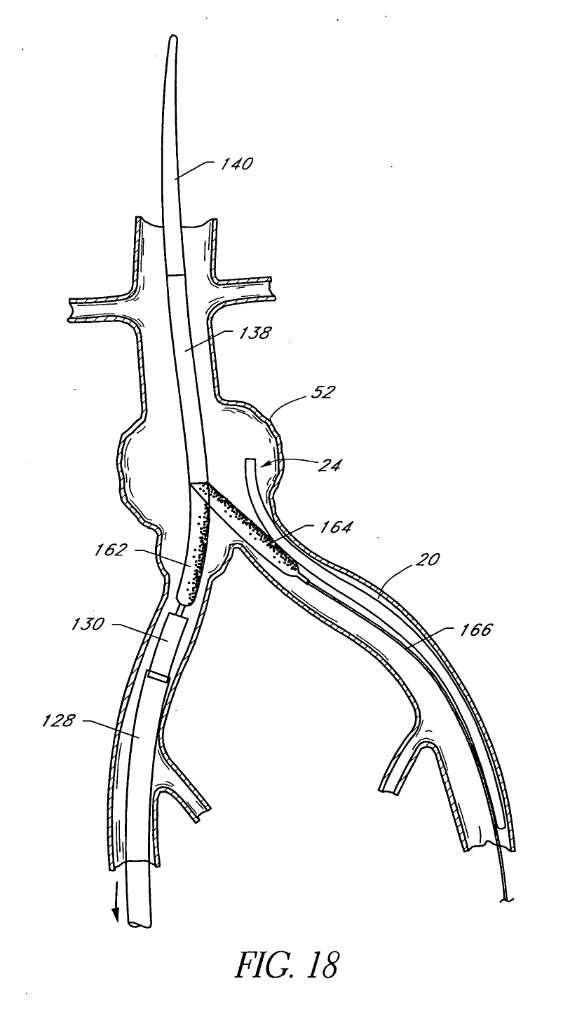

With reference now to FIGS. 17-23, one embodiment of use for decompressing an aneurysm located generally at or near the bifurcation of the lower abdominal aorta and the ipsilateral and contralateral iliac arteries will now be described. The free end of a contralateral guidewire 166 is preferably advanced through a first lumen of a dual lumen catheter as is described in U.S. Pat. No. 6,440,161, issued on Aug. 27, 2002, the disclosure of which is hereby incorporated herein in its entirety. The deployment catheter 120 is thereafter percutaneously inserted into the first puncture, and advanced along guidewire (e.g. 0.035 inch) through the ipsilateral iliac and into the aorta. As the deployment catheter 120 is transluminally advanced, slack produced in the contralateral guidewire 166 is taken up by proximally withdrawing the guidewire 166 from the second percutaneous access site. In this manner, the deployment catheter 120 is positioned in the manner generally illustrated in FIG. 17. Before or after positioning the deployment catheter 120, the distal end of the aspiration catheter 20 may be positioned in the aneurysm 52. In the illustrated embodiment, the aspiration catheter 20 is inserted through the second puncture and through the contralateral iliac. In such an embodiment, the aspiration catheter 120 may be inserted over its own guidewire (not shown) or the contralateral guidewire 166. In other embodiments, the aspiration catheter 20 may be inserted through the first puncture site through the ipsilateral iliac adjacent the deployment catheter 120. In such an embodiment, the aspiration catheter 20 may be inserted over the ipsilateral guidewire or a separate guidewire.

With the aspiration catheter 20 positioned in the aneurysm 52, the graft is deployed. In the illustrated embodiment, this is accomplished by proximally withdrawing the outer sheath 128 while maintaining the axial position of the overall deployment catheter 120, thereby releasing the first and second iliac branches of the graft 150. Proximal advancement of the deployment catheter 120 and contralateral guidewire 166 can then be accomplished, to position the iliac branches of the graft 150 within the iliac arteries as illustrated.

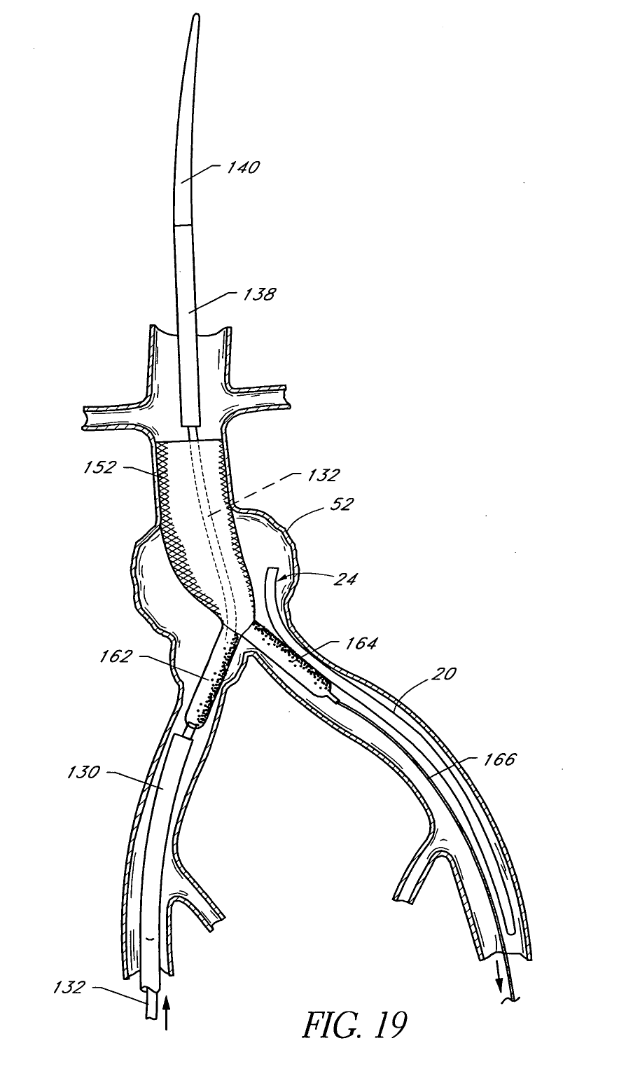

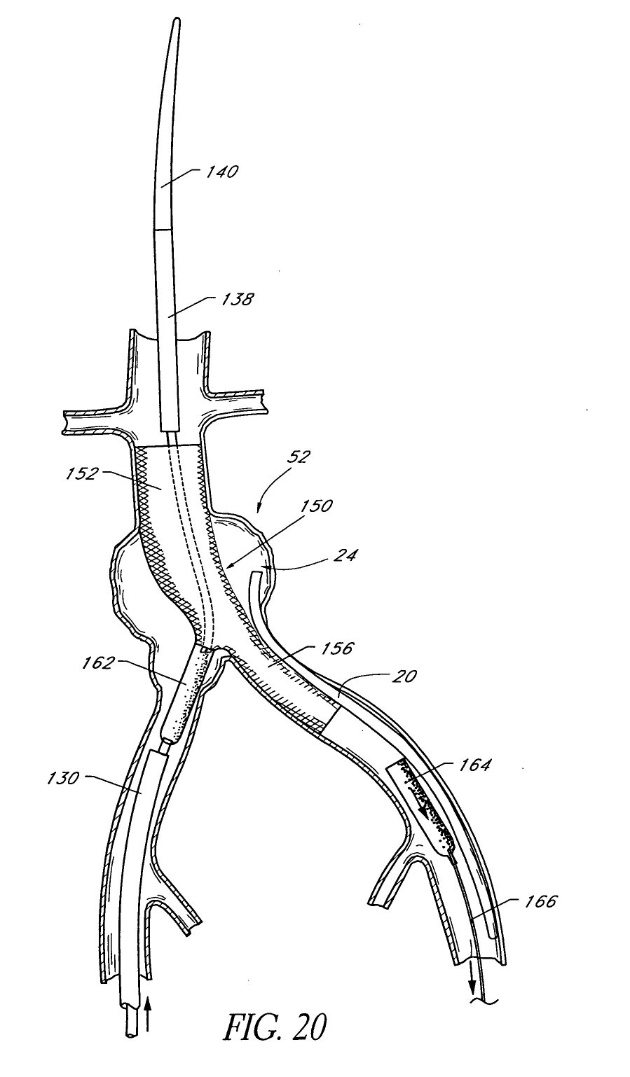

Referring to FIG. 19, the central core 132 is distally advanced thereby distally advancing the distal housing 138. This exposes the aortic trunk 152 of the graft 150, which deploys into its fully expanded configuration within the aorta. As illustrated in FIG. 20, the contralateral guidewire 166 is thereafter proximally withdrawn, thereby by proximally withdrawing the second sheath 164 from the contralateral iliac branch 156 of the graft 150. The contralateral branch 156 of the graft 150 thereafter self expands to fit within the iliac artery. The guidewire 166 and sheath 164 may thereafter be proximally withdrawn and removed from the patient, by way of the second percutaneous access site. As shown in FIG. 20, in this embodiment, the aspiration catheter 20 is positioned on the outside of the contralateral branch 156 of the graft 150 while the distal end 24 remains in the aneurysm 52.

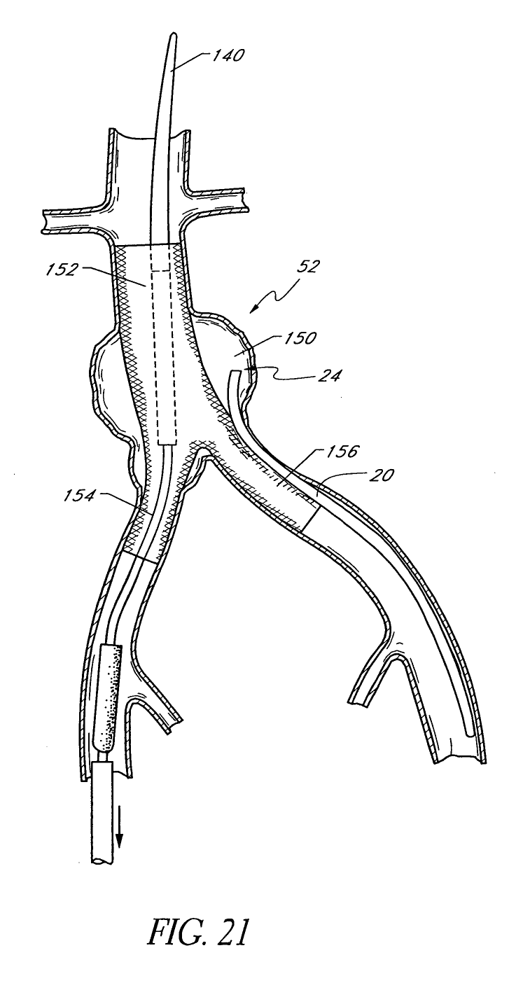

Thereafter, the deployment catheter 120 may be proximally withdrawn to release the ipsilateral branch 154 of the graft 150 from the first tubular sheath 162 as shown in FIG. 21. Following deployment of the ipsilateral branch 154 of the prosthesis 150, a central lumen through the aortic trunk 152 and ipsilateral branch 154 is sufficiently large to permit proximal retraction of the deployment catheter 120 through the deployed bifurcated graft 150. The deployment catheter 120 may thereafter be proximally withdrawn from the patient by way of the first percutaneous access site.

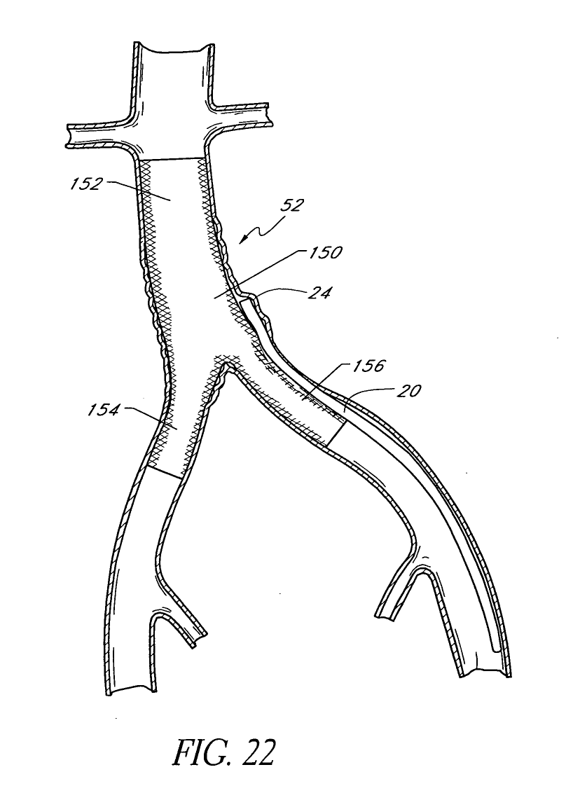

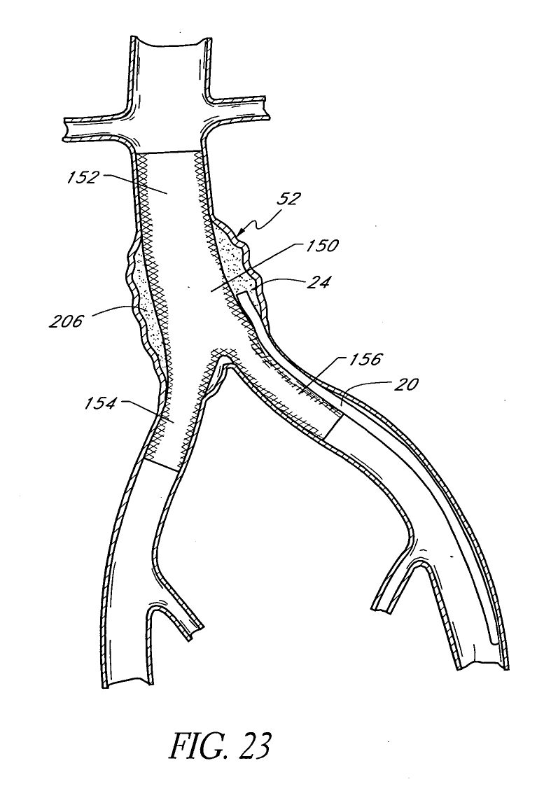

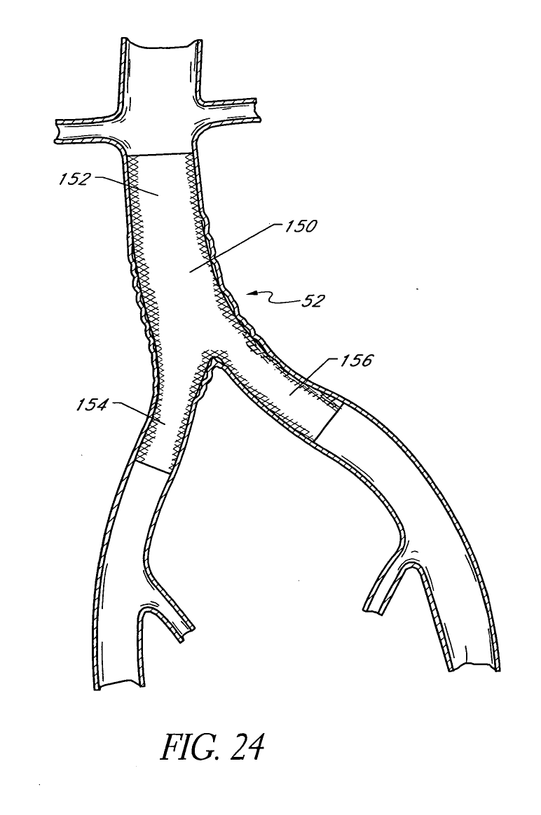

With the aneurysm isolated 52, material may be aspirated from the aneurysm through the aspirating catheter 20. As the aneurysm 52 is decompressed, the volume of the aneurysm sac is reduced as shown in FIG. 22. In this manner, the sac is pulled closer to the graft 150 as the volume of the sac is reduced. As mentioned above, in certain embodiments, before or after aspiration, the aspiration catheter 20 may also be used to deliver a medical agent into the isolated portion of the aneurysm. For example, in one embodiment of use, an embolization material 206 may be delivered to the decompressed aneurysm sac 52 to further reduce the possibility of endoleaks. (see FIG. 23). The aspiration catheter 20 may then be removed leaving the graft 150 in place (see FIG. 24, showing the sac without an embolization material). In the illustrated embodiment, the contralateral branch 156 of the graft 150 is a self-expandable graft such that the graft expands to occupy the space vacated by the catheter 20.

As mentioned above, decompressing the aneurysm may have several advantageous results. For example, the wall stress of the aneurysm is generally proportional to the diameter of the sac. Accordingly, the reduction of sac diameter may reduce local wall stress in the aorta. Decompressing the aneurysm may also increase of contact area between the graft and the vessel wall to increase sealing and in-growth. In addition, removing material from the aneurysm may create a void for injection of a medical agent (e.g., a embolization agent). In contrast, if material is not removed from the sac, injection of an agent could increase the sac size and subsequently aortic wall stresses.

In the illustrated embodiment, the aneurism 52 is isolated with a self-expanding bifurcated graft as described above. However, it should be appreciated that the other self-expanding grafts may also be used including bifurcated grafts in which one or more portions of the grafts are assembled within the patient (e.g., see U.S. Pat. No. 6,582,458, which is hereby incorporated by reference in its entirety herein). In addition, self expanding straight grafts may also be used to isolate the aneurysm (see e.g., U.S. Pat. No. 6,077,296, which is hereby incorporated by reference herein in its entirety). In still other embodiments, the graft may expanded by an expandable device (e.g., a balloon).

As mentioned above, U.S. Pat. Nos. 6,582,458, 6,077,296, 6,197,049, 6,090,128, 6,261,316, 6,440,161, U.S. Application Publication No. 2002/0052644 and International Publication No. 02/39888 and the entire disclosure of all of these patents is hereby incorporated by reference herein.

As mentioned above, in some embodiments, the agent can be delivered into the sac in multiple injections. These injections can be are alternated with injections of a contrast agent. In such an embodiment, both the agent and the contrast agent can be injected into the sac through the same catheter 20 and/or through separate catheters (not shown). After and/or during injection of the contrast agent, a sacogram can be performed to visualize the extent to which the sac is filled with the agent and to which endoleaks are being prevented or reduced. As explained below, in such embodiments, it is generally preferred that the sac is filled from its distal end to its proximal end (with respect the graft as described herein). Thus, in between injections and/or during injection of the agent, the catheter 20 can be proximally withdrawn to progressively fill the proximal portions of the sac. The injections of contrast agent are preferably done after the agent has had time to stabilize.

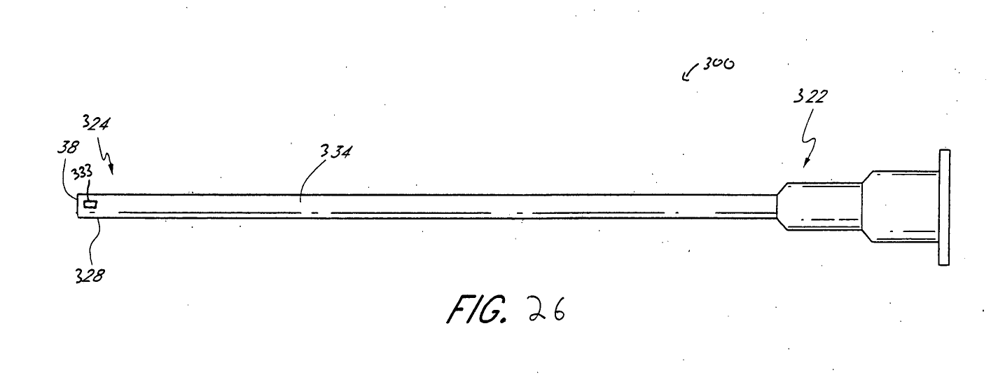

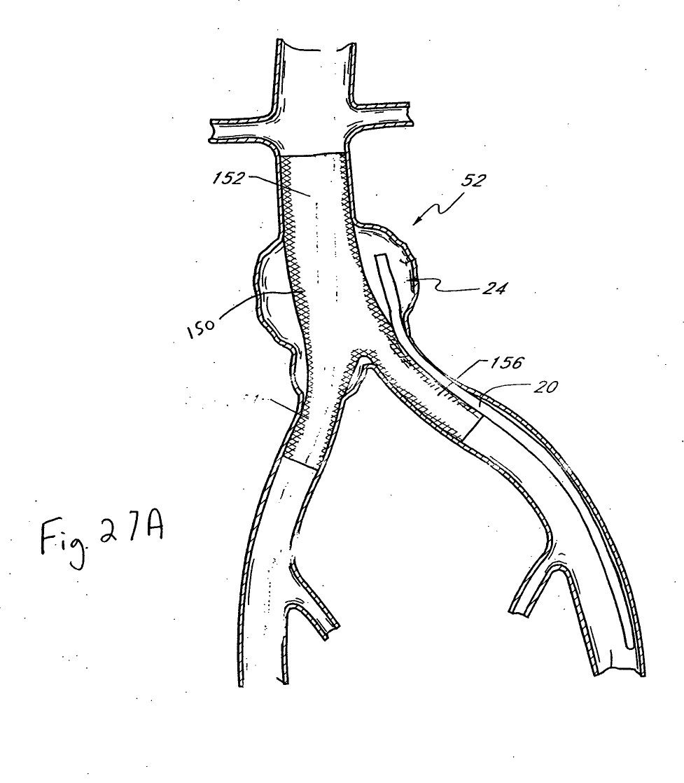

FIG. 26 illustrates an introducer sheath 300 that can be used in the embodiments described herein. The introducer sheath 300 comprises a distal end 324 and an elongate flexible tubular body 326 extending therebetween. A lumen (not shown) extends axially through the tubular body 326 between a proximal access port 330 and a distal access port 332. The distal end 324 of the catheter 320 preferably also includes a radiopaque marker 333, which can be used to position the distal end 324 within the sac. The distal end 324 of the sheath 300 preferably includes a smooth non-reactive coating that minimizes or reduces adhesion of agent that is injected into the sac.

The introducer sheath 300 can be inserted percutaneously from the femoral artery as described above until the distal end 324 is placed in the aneurysm sac. The graft can then be placed over the introducer sheath 300 as described above. The aspiration catheter 20 or other catheters described herein for aspiration and/or delivering an agent to the sac can exchanged through the introducer sheath 300 to perform one or more of the methods described herein. Thus, in the description herein, when reference is made to a catheter it should be appreciated that the catheter can be placed within the introducer sheath.

As a further alternative, the sheath 300 can be positioned in the sac through the use of separate percutaneous tissue tract, formed through the chest wall as described above. In either alternative, the sheath 300 is preferably positioned in the sac before the graft is fully deployed. In addition, the sheath 300 can be placed in the sac using a trans-lumbar access after placement of the stent graft.

There are three general types of endoleaks identified within the aneurysm sac. Type I endoleaks can represent failure of the attachment of the graft to the vascular site. Type III endoleaks can represent separation of device components. There is general agreement that these failure modes necessitate urgent repair because blood flow and systemic pressure will continue to be transmitted into the aneurysm sac, putting the patient at continued risk for aneurysm enlargement and rupture. Type II endoleaks can be caused when flow enters the aneurysm sac after graft placement through aortic branch vessels (e.g., the lumbar arteries).

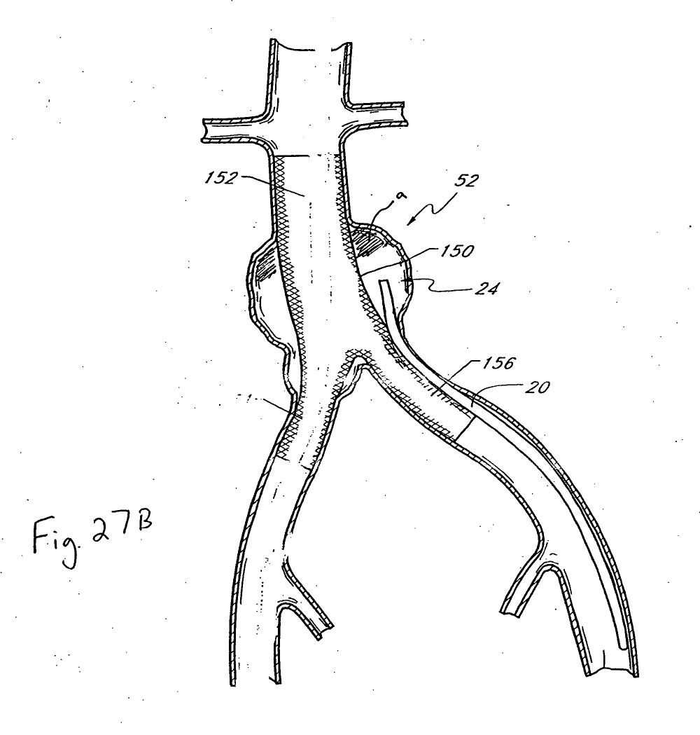

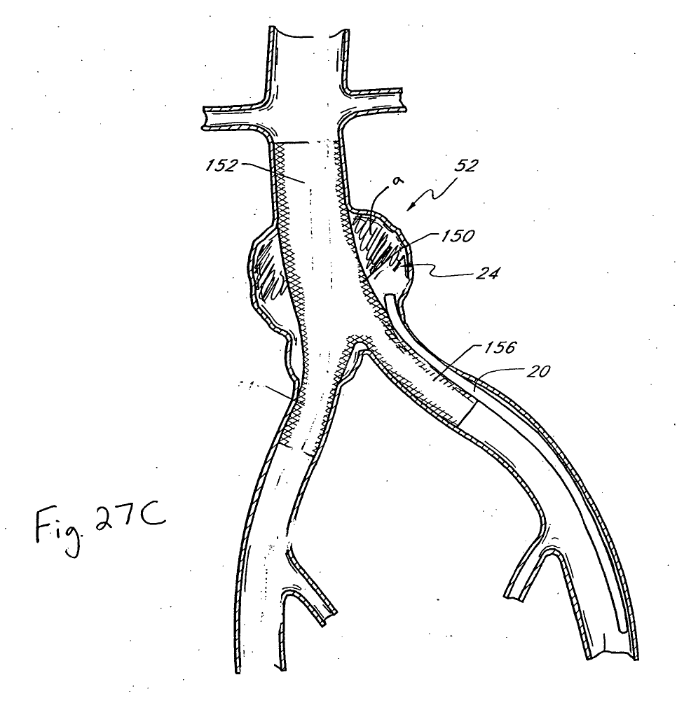

With reference to FIGS. 27-C, in one embodiment, after the graft 150 is placed, an aortagram is performed to identify potential type II endoleaks. Then, using an introducer sheath 300 or a catheter 20 (optionally inserted within an introducer sheath 300), a sacogram is performed by injecting contrast medium into the sac 52. During the sacogram, the position of the lumbar arteries (not shown), which emerge from the sac 52, can be marked on the screen used for the sacogram.

The distal end 24 of the catheter is preferably positioned at or near the location of the distal most lumbar arteries (not shown). An embolizing material or agent is then injected into the sac 52. The injection can be monitored on the screen used in the sacogram. In one embodiment, the agent is injected until the agent enters or substantially closes the openings to the lumbar arteries. A sacogram can then be performed by injecting contrast agent into the sac 52 through the catheter 20. If the sacogram indicates that distal lumbar arteries are still patent, more agent can be injected into the sac 52. The sacogram is preferably performed after the agent has had time to stabilize.

With reference to FIG. 26B, the catheter 20 is withdrawn until the tip 24 is positioned generally in the central region or blood pool of the sac 52. The agent a is again injected into the sac at a preferably slow rate while the injection is monitored on the screen. During this step, the catheter 20 can be withdrawn proximally to more uniformly disperse the agent within the sac 52. In addition, care should be taken not to inject too much agent such that the graft 150 begins to compress.

After the central region is filled, a sacogram is performed. If the proximal lumbar arteries are still patent, the catheter 20 is withdrawn until the distal end 24 is positioned near or at the location of the proximal lumbar arteries. See FIG. 27C. The agent α is again injected into the sac 52. The injection can be monitored on the screen used in the sacogram. In one embodiment, the agent is injected until the agent enters or substantially closes the openings to the proximal lumbar arteries. After the agent α has solidified, a sacogram can be performed to determined if the proximal arteries are still patent. If they are, more agent can be injected into the sac 52. An aortogram can be performed to confirm the absence of endoleaks. If the graft has partially collapsed due to excessive injection of the agent α into the sac, the graft 150 can be expanded with a balloon catheter to reestablish a patent lumen. Another aortogram can be then be performed to verify that the graft is patent.

In the method described above, the introducer sheath 300 of FIG. 26 can be used. In such an embodiment, the sheath 300 is positioned in the sac 52 before the graft is fully deployed. The sheath 300 can be used to inject contrast agent and to facilitate the passage of the catheter 20 into the sac 52. For example, it may be advantageous to use multiple catheters 20 to inject the agent into the sac 20 in multiple injections. This is because the agent may solidify in the catheter 20 between injections. The sheath 300 facilitates the exchange of multiple injection catheters 20.

Many embolization agents take several seconds to several minutes to stabilize. Some of these embolization agents also release gas during the curing process. This can cause the pressure in the sac to increase during the stabilization process, which can lead to the uncontrolled release of agent into the lumbar arteries. This can be overcome or reduced by maintaining the pressure in the sac such that is lower than in the spinal artery thereby creating a negative pressure gradient that will create retrograde flow from the spinal artery to the sac preventing agent from traveling into the lumbar arteries. Venting can be provided in several different manners. For example, in one embodiment, a second catheter is inserted into the sac to provide venting to the atmosphere. In another embodiment, the catheter used to deliver the agent includes a separate aspiration or venting lumen. In either embodiment, a vacuum can be applied to the sac through the venting apparatus to elevate the blood pressure of the spinal artery above that of the sac. In these embodiments, the venting or vacuum can be applied using the introducer sheath 300 or through a catheter inserted therein. In another embodiment the systemic pressure in the spinal region may be raised above that of the sac.

Various combinations and sub-combinations of the components described above can be packaged, sold and/or used together as a kit. For example, in one embodiment, a kit for treating a patient with a vascular aneurysm comprises a vascular graft configured according to the embodiments described above. The kit also includes deployment catheter, which can be configured as described above, to deploy the vascular graft within the aneurysm to isolate a portion thereof. The kit also includes an aspiration catheter as described above. In one modified embodiment, the kit includes an agent (e.g., embolization material) that is configured to be inserted into the isolated portion of the aneurism. A measurement device as described above can also be provided to measure the amount of material aspirated through the aspiration catheter and/or the amount of agent injected into the aneurism.

While a number of preferred embodiments of the invention and variations thereof have been described in detail, other modifications and methods of using and medical applications for the same will be apparent to those of skill in the art. Accordingly, it should be understood that various applications, modifications, and substitutions may be made of equivalents without departing from the spirit of the invention or the scope of the claims.

What is claimed is:

1. A method of treating a patient, comprising the steps of:
identifying a vascular aneurysm;
positioning a prosthesis across the aneurysm, to isolate at least a portion of the aneurysm from an adjacent vessel; and
removing material from the isolated portion of the aneurysm; and
introducing an embolization material through an introducing catheter into a first portion of the isolated portion;
repositioning the introducing catheter;
introducing the embolization material through the introducing catheter into a second portion of the isolated portion.

2. The method of claim 1, wherein a contrast agent is introduced of the aneurysm between the steps of introducing the embolization material into the first portion of and the second portion of isolated portion.

3. The method of claim 1, wherein the first portion is located distally as compared to the second portion.

4. The method of claim 1, wherein the step of repositioning the introducing catheter comprises proximally withdrawing the introducing catheter.

5. A method of treating a patient, comprising the steps of:
identifying a vascular aneurysm;
positioning a prosthesis across the aneurysm, to isolate at least a portion of the aneurysm from an adjacent vessel; and
removing material from the isolated portion of the aneurysm;
introducing an embolization material into the isolated portion of the aneurysm; and
venting the isolated portion of the aneurysm while the embolization material is introduced into the isolated portion of the aneurysm.

6. A method of treating a patient, comprising the steps of:
identifying a vascular aneurysm;
positioning a prosthesis across the aneurysm, to isolate at least a portion of the aneurysm from an adjacent vessel; and
removing material from the isolated portion of the aneurysm;
introducing an embolization material into the isolated portion of the aneurysm; and
applying a vacuum to the isolated portion of the aneurysm while the embolization material is introduced into the isolated portion of the aneurysm.

7. A kit for treating a patient with a vascular aneurysm, the kit comprising:
a vascular graft configured to isolate at least a portion of the aneurysm;
a deployment catheter configured to deploy the vascular graft within the aneurysm; and
an delivery catheter comprising an elongate body that defines an delivery lumen configured to deliver an embolization material.
and introducer sheath configured to receive the delivery catheter.

8. A kit for treating a patient with a vascular aneurysm, the kit comprising:
a vascular graft configured to isolate at least a portion of the aneurysm;
a deployment catheter configured to deploy the vascular graft within the aneurysm; and
an delivery catheter comprising an elongate body that defines an delivery lumen configured to deliver an embolization material and an aspiration lumen configured to provide a vent to the vascular aneurysm.
